(19) United States
(12) Patent Application Publication
(10) Pub. No.: US 2024/0219380 A1
(43) Pub. Date: Jul. 4, 2024

(54) METHODS FOR MEASURING POLY A TAIL LENGTH

(71) Applicant: TRANSLATE BIO, INC., Lexington, MA (US)

(72) Inventors: Jonathan ABYSALH, Lexington, MA (US); Anusha DIAS, Lexington, MA (US); Jeffrey S. DUBINS, Lexington, MA (US); Jorel E. VARGAS, Lexington, MA (US)

(21) Appl. No.: 18/557,446

(22) PCT Filed: Apr. 29, 2022
(86) PCT No.: PCT/US2022/026913

§ 371 Date: Oct. 26, 2023

Related U.S. Application Data
(60) Provisional application No. 63/181,488, filed on Apr. 29, 2021.

(51) Int. Cl. G01N 33/53

(52) U.S. Cl. CPC G01N 33/5308

(57) ABSTRACT

The present invention provides, among other things, methods of measuring homopolymeric nucleotide lengths in a nucleic acid, including mRNA. In some aspects, provided herein is a method of measuring poly A tail length in mRNA comprising binding of mRNA with a minor-groove binding dye, followed by ribonuclease digestion and capillary electrophoresis.

### CROSS-REFERENCE TO RELATED APPLICATIONS

This application claims priority to U.S. Provisional Patent Application Ser. No. 63/181,488, filed on Apr. 29, 2021, which is incorporated by reference herein in its entirety for all purposes.

### INCORPORATION-BY-REFERENCE OF SEQUENCE LISTING

The contents of the text file named “MRT-2220WO1_ST25.txt”, which was created on Apr. 15, 2022 and is 688 bytes in size, is hereby incorporated by reference in its entirety.

### BACKGROUND

Messenger RNA therapy (MRT) is a promising approach to treat a variety of diseases. MRT involves administration of messenger RNA (mRNA) to a patient in need of the therapy. The administered mRNA produces a protein or peptide encoded by the mRNA within the patient's body. mRNA is typically synthesized using in vitro transcription systems (IVT) which involve enzymatic reactions by RNA polymerases. An IVT synthesis process is usually followed by reaction(s) for the addition of a 5′-cap (capping reaction) and a 3′-poly A tail (polyadenylation).

Effective mRNA therapy requires delivery of mRNA to the patient and efficient production of the protein encoded by the mRNA within the patient's body. The 5′ cap and 3′ poly A tail play a role in optimizing mRNA delivery and protein production in vivo. The cap at the 5′ end prevents degradation and improves translation. The poly A tail at the 3′ end protects the mRNA from exonuclease degradation and improves integrity and stability of mRNA for mRNA therapeutics.

### SUMMARY OF THE INVENTION

Assessment of poly A tail length is performed as a quality control measurement for mRNA therapeutics. Accurate poly A tail length measurement is also used for establishing dose by accurately quantifying the stable mRNA therapeutic that is intact, full-length and translated into functional protein upon delivery. Currently available methods of determining poly A tail length have certain disadvantages, including low accuracy among others.

Current methods for determining poly A tail length include, for example, measurements using poly A binding protein assays, that require a poly A tail that is long enough to bind at least 4 monomers of poly A binding protein where each monomer binds to stretches of approximately 38 nucleotides. Other methods include using a ligation mediated poly A measurement that is based on a PCR assay that requires a reverse transcription step and cDNA synthesis from oligo dT primers, which can be inaccurate for longer poly A tails. Another method, an RNase H based method involves removing the poly A tail from the mRNA of interest, which is not suitable for mRNA therapeutics requiring an intact poly A tail.

Methods for measuring mRNA tail length for mRNA therapeutics include, for example, a capillary electrophoresis (CE) method and an RNase A method.

The CE method does not require enzymatic digestion and is accomplished in a short run time of about 1 hour. It can be employed in a high-throughput manner where up to 48 samples can be processed simultaneously. However, as tail lengths increase, the measured tail lengths are often inaccurate as retention times in capillary electrophoresis shift. The CE method commonly employs intercalating dyes, such as the Agilent™ intercalating dye, which result in a weak signal with homopolymeric stretches, including poly A tails.

The RNase A gel method requires enzymatic digestions that allows for specific degradation after C and Us, leaving the poly A tails to be measured. It provides a reproducible and consistent measure of tail lengths. However, it is requires a 30 minute enzymatic digestion step and a gel run time of 2 hours and 30 minutes. This method is performed in a low-throughput manner of about 10 samples/gel.

The present invention provides, among other things, a method of accurately measuring poly A tail length in an mRNA sample in a rapid and high-throughput manner. The present invention is based, in part, on the surprising and unexpected finding that binding of a minor groove binding dye with mRNA, followed by ribonuclease (RNase) digestion and capillary electrophoresis (CE) provides an accurate method of determining poly A tail length of mRNA. In some embodiments, one or more steps of the method is automated. In some embodiments, the method is high throughput.

Accurately measuring long poly A tail lengths of between 50 to 200 or more nucleotides is challenging using current methods. Provided herein is a method that can reliably and accurately measure long poly A tail lengths of 50 or more, 100 or more, 150 or more or 200 or more nucleotides in an efficient and high-throughput manner. In some embodiments, the measured poly A tail lengths using this method are equal to a theoretical tail length. In some embodiments, the measured tail lengths are 100% accurate. In some embodiments, the measured poly A tail lengths using this method approach the theoretical tail length. In some embodiments, the measured poly A tail lengths are greater than 90%, greater than 91%, greater than 92%, greater than 93%, greater than 94%, greater than 95%, greater than 96%, greater than 97%, greater than 98% or greater than 99% accurate. In some embodiments, the measured poly A tail lengths are greater than 50%, greater than 60%, greater than 70%, greater than 80%, or greater than 90% accurate.

Capillary electrophoresis (CE) is typically carried out using intercalating dyes. In some embodiments, the intercalating dye is Agilent™ intercalating dye. The present invention is based, in part, on the unexpected results obtained by using minor groove binding dyes in capillary electrophoresis. Accordingly, in some embodiments, the dye used in the methods described herein is a minor groove binding dye. In some embodiments, the minor groove binding dye is Sybr Gold™.

Poly A tails are helical and are made of planar stacked bases. Minor groove binding dyes selectively bind non-covalently to RNA via hydrogen bonding and hydrophobic interactions. Without wishing to be bound by any particular theory, it is contemplated that minor groove binding dyes, including Sybr Gold™, can bind to the planar structure formed by poly A tails due to the interaction not being dependent on base stacking. Intercalating dyes such as Agilent™ intercalating dye produce a dampened poly A signal because intercalating dyes cannot properly pi-stack into the planar structure created by poly A tails, resulting in inaccurate poly A tail length measurements. In contrast, Sybr Gold™ binds non-covalently to the helical structure of the poly A tail by binding to the minor grooves formed by the secondary structure of the single strand, providing a method of accurately measuring poly A tail length (FIG. 1B).

In some aspects, provided herein is a method of measuring poly A tail length in an mRNA sample, the method comprising: (a) contacting the mRNA sample with a minor-groove binding dye; (b) incubating the mRNA sample from (a) with one or more ribonucleases (RNase); and (c) assaying the sample from (b) by capillary electrophoresis (CE) to determine the poly A tail length of the mRNA.

In some embodiments, provided herein is a method, wherein the minor-groove binding dye is Sybr Gold™, a Hoechst dye, or 4′,6-diamidino-2-phenylindole (DAPI). In some embodiments, the minor-groove binding dye is Sybr Gold™. In some embodiments, the minor-groove binding dye is a Hoechst dye. In some embodiments, the minor-groove binding dye is 4′,6-diamidino-2-phenylindole (DAPI).

In some embodiments, provided herein is a method, wherein the mRNA sample is incubated with one or more RNases selected from RNase A and RNase T1. In some embodiments, the one or more RNases is RNase A. In some embodiments, the one or more RNases is RNase T1. In some embodiments, the one or more RNases comprises RNase A1 and RNase T1. RNase A degrades RNA after C and U residues, while RNase T1 degrades after G residues. Digestion with RNase A and RNase T1 ensures that only poly A tails remain.

In some embodiments, provided herein is a method wherein the capillary electrophoresis (CE) is coupled with a fluorescence-based detection.

In some embodiments, provided herein is a method wherein the capillary electrophoresis (CE) is coupled with UV absorption spectroscopy detection.

In some embodiments, the mRNA sample is incubated with one or more ribonucleases (RNase) is for about 15 minutes, 30 minutes, 45 minutes, or 60 minutes. In some embodiments, the mRNA sample is incubated with one or more ribonucleases (RNase) is for about 15 minutes. In some embodiments, the mRNA sample is incubated with one or more ribonucleases (RNase) is for about 30 minutes. In some embodiments, the mRNA sample is incubated with one or more ribonucleases (RNase) is for about 45 minutes. In some embodiments, the mRNA sample is incubated with one or more ribonucleases (RNase) is for about 60 minutes.

In some embodiments, the poly A tail length is 25 nucleotides or more.

In some embodiments, the poly A tail length is between 50 nucleotides and 5,000 nucleotides. In some embodiments, the poly A tail length is about 50 nucleotides. In some embodiments, the poly A tail length is about 100 nucleotides. In some embodiments, the poly A tail length is about 150 nucleotides. In some embodiments, the poly A tail length is about 200 nucleotides. In some embodiments, the poly A tail length is about 250 nucleotides. In some embodiments, the poly A tail length is about 300 nucleotides. In some embodiments, the poly A tail length is about 350 nucleotides. In some embodiments, the poly A tail length is about 400 nucleotides. In some embodiments, the poly A tail length is about 450 nucleotides. In some embodiments, the poly A tail length is about 500 nucleotides. In some embodiments, the poly A tail length is about 550 nucleotides. In some embodiments, the poly A tail length is about 600 nucleotides. In some embodiments, the poly A tail length is about 650 nucleotides. In some embodiments, the poly A tail length is about 700 nucleotides. In some embodiments, the poly A tail length is about 750 nucleotides. In some embodiments, the poly A tail length is about 800 nucleotides. In some embodiments, the poly A tail length is about 850 nucleotides. In some embodiments, the poly A tail length is about 900 nucleotides. In some embodiments, the poly A tail length is about 950 nucleotides. In some embodiments, the poly A tail length is about 1000 nucleotides. In some embodiments, the poly A tail length is about 1200 nucleotides. In some embodiments, the poly A tail length is about 1400 nucleotides. In some embodiments, the poly A tail length is about 1600 nucleotides. In some embodiments, the poly A tail length is about 1800 nucleotides. In some embodiments, the poly A tail length is about 2000 nucleotides. In some embodiments, the poly A tail length is about 2200 nucleotides. In some embodiments, the poly A tail length is about 2400 nucleotides. In some embodiments, the poly A tail length is about 2600 nucleotides. In some embodiments, the poly A tail length is about 2800 nucleotides. In some embodiments, the poly A tail length is about 3000 nucleotides. In some embodiments, the poly A tail length is about 3200 nucleotides. In some embodiments, the poly A tail length is about 3400 nucleotides. In some embodiments, the poly A tail length is about 3600 nucleotides. In some embodiments, the poly A tail length is about 3800 nucleotides. In some embodiments, the poly A tail length is about 4000 nucleotides. In some embodiments, the poly A tail length is about 4200 nucleotides. In some embodiments, the poly A tail length is about 4400 nucleotides. In some embodiments, the poly A tail length is about 4600 nucleotides. In some embodiments, the poly A tail length is about 4800 nucleotides. In some embodiments, the poly A tail length is about 5000 nucleotides.

In some embodiments, the poly A tail length is 50 or more nucleotides. In some embodiments, the poly A tail length is 100 or more nucleotides. In some embodiments, the poly A tail length is 150 or more nucleotides. In some embodiments, the poly A tail length is 200 or more nucleotides.

In some embodiments, the poly A tail length is between 100 nucleotides and 1,500 nucleotides. In some embodiments, the poly A tail length is about 100 nucleotides. In some embodiments, the poly A tail length is about 200 nucleotides. In some embodiments, the poly A tail length is about 300 nucleotides. In some embodiments, the poly A tail length is about 400 nucleotides. In some embodiments, the poly A tail length is about 500 nucleotides. In some embodiments, the poly A tail length is about 600 nucleotides. In some embodiments, the poly A tail length is about 700 nucleotides. In some embodiments, the poly A tail length is about 800 nucleotides. In some embodiments, the poly A tail length is about 900 nucleotides. In some embodiments, the poly A tail length is about 1000 nucleotides.

In some embodiments, the poly A tail length is between 250 nucleotides and 500 nucleotides. In some embodiments, the poly A tail length is about 250 nucleotides. In some embodiments, the poly A tail length is about 260 nucleotides. In some embodiments, the poly A tail length is about 270 nucleotides. In some embodiments, the poly A tail length is about 280 nucleotides. In some embodiments, the poly A tail length is about 290 nucleotides. In some embodiments, the poly A tail length is about 300 nucleotides. In some embodiments, the poly A tail length is about 310 nucleotides. In some embodiments, the poly A tail length is about 320 nucleotides. In some embodiments, the poly A tail length is about 330 nucleotides. In some embodiments, the poly A tail length is about 340 nucleotides. In some embodiments, the poly A tail length is about 350 nucleotides. In some embodiments, the poly A tail length is about 360 nucleotides. In some embodiments, the poly A tail length is about 370 nucleotides. In some embodiments, the poly A tail length is about 380 nucleotides. In some embodiments, the poly A tail length is about 390 nucleotides. In some embodiments, the poly A tail length is about 400 nucleotides. In some embodiments, the poly A tail length is about 410 nucleotides. In some embodiments, the poly A tail length is about 420 nucleotides. In some embodiments, the poly A tail length is about 430 nucleotides. In some embodiments, the poly A tail length is about 440 nucleotides. In some embodiments, the poly A tail length is about 450 nucleotides. In some embodiments, the poly A tail length is about 460 nucleotides. In some embodiments, the poly A tail length is about 470 nucleotides. In some embodiments, the poly A tail length is about 480 nucleotides. In some embodiments, the poly A tail length is about 490 nucleotides. In some embodiments, the poly A tail length is about 500 nucleotides.

In some embodiments, one or more steps of the method is automated.

In some embodiments, incubating the mRNA sample with one or more ribonucleases (RNase) is automated.

In some embodiments, the minor-groove binding dye non-covalently binds to single-stranded RNA (ssRNA).

In some embodiments, the minor-groove binding dye is not an intercalating dye.

In some aspects, provided herein is a method of measuring poly A tail length in an mRNA, the method comprising: (a) contacting the mRNA sample with Sybr Gold™ minor-groove binding dye; (b) incubating the mRNA sample from (a) with RNaseA and RNase T1; and (c) assaying the sample from (b) by capillary electrophoresis (CE) to determine the poly A tail length of the mRNA.

In some embodiments, the method comprises capillary electrophoresis (CE) coupled with a fluorescence-based detection.

In some embodiments, the method comprises capillary electrophoresis (CE) coupled with UV absorption spectroscopy detection.

In some embodiments, the method comprises incubating the mRNA sample with RNaseA and RNase T1 is for about 15 minutes, 30 minutes, 45 minutes, or 60 minutes. In some embodiments, incubating the mRNA sample with RNase A and RNase T1 is for about 15 minutes. In some embodiments, incubating the mRNA sample with RNase A and RNase T1 is for about 30 minutes. In some embodiments, incubating the mRNA sample with RNase A and RNase T1 is for about 45 minutes. In some embodiments, incubating the mRNA sample with RNaseA and RNase T1 is for about 60 minutes.

In some embodiments, the poly A tail length is 25 or more nucleotides, 50 or more nucleotides, 100 or more nucleotides, 150 or more nucleotides, or 200 or more nucleotides. In some embodiments, the poly A tail length is 25 or more nucleotides. In some embodiments, the poly A tail length is 50 or more nucleotides. In some embodiments, the poly A tail length is 100 or more nucleotides. In some embodiments, the poly A tail length is 150 or more nucleotides. In some embodiments, the poly A is 200 or more nucleotides.

In some embodiments, one or more steps of the method is automated.

In some embodiments, the method is high throughput.

In some aspects, provided herein is a method of measuring homopolymeric nucleotide length in an mRNA sample, the method comprising: (a) contacting the mRNA sample with a minor-groove binding dye; (b) incubating the mRNA sample from (a) with one or more ribonucleases (RNase); and (c) assaying the sample from (b) by capillary electrophoresis (CE) to determine the homopolymeric nucleotide length of the mRNA.

In some embodiments, the homopolymeric nucleotide length is 25 nucleotides or more.

In some embodiments, the homopolymeric nucleotide length is between 50 nucleotides and 5,000 nucleotides. In some embodiments, the homopolymeric nucleotide length is 50 nucleotides. In some embodiments, the homopolymeric nucleotide length is 100 nucleotides. In some embodiments, the homopolymeric nucleotide length is 150 nucleotides. In some embodiments, the homopolymeric nucleotide length is 200 nucleotides. In some embodiments, the homopolymeric nucleotide length is 250 nucleotides. In some embodiments, the homopolymeric nucleotide length is 300 nucleotides. In some embodiments, the homopolymeric nucleotide length is 350 nucleotides. In some embodiments, the homopolymeric nucleotide length is 400 nucleotides. In some embodiments, the homopolymeric nucleotide length is 450 nucleotides. In some embodiments, the homopolymeric nucleotide length is 500 nucleotides. In some embodiments, the homopolymeric nucleotide length is 550 nucleotides. In some embodiments, the homopolymeric nucleotide length is 600 nucleotides. In some embodiments, the homopolymeric nucleotide length is 650 nucleotides. In some embodiments, the homopolymeric nucleotide length is 700 nucleotides. In some embodiments, the homopolymeric nucleotide length is 750 nucleotides. In some embodiments, the homopolymeric nucleotide length is 800 nucleotides. In some embodiments, the homopolymeric nucleotide length is 850 nucleotides. In some embodiments, the homopolymeric nucleotide length is 900 nucleotides. In some embodiments, the homopolymeric nucleotide length is 950 nucleotides. In some embodiments, the homopolymeric nucleotide length is 1000 nucleotides.

In some embodiments, the homopolymeric nucleotide length is 1100 nucleotides. In some embodiments, the homopolymeric nucleotide length is 1200 nucleotides. In some embodiments, the homopolymeric nucleotide length is 1300 nucleotides. In some embodiments, the homopolymeric nucleotide length is 1400 nucleotides. In some embodiments, the homopolymeric nucleotide length is 1500 nucleotides. In some embodiments, the homopolymeric nucleotide length is 1600 nucleotides. In some embodiments, the homopolymeric nucleotide length is 1700 nucleotides. In some embodiments, the homopolymeric nucleotide length is 1800 nucleotides. In some embodiments, the homopolymeric nucleotide length is 1900 nucleotides. In some embodiments, the homopolymeric nucleotide length is 2000 nucleotides. In some embodiments, the homopolymeric nucleotide length is 2100 nucleotides. In some embodiments, the homopolymeric nucleotide length is 2200 nucleotides. In some embodiments, the homopolymeric nucleotide length is 2300 nucleotides. In some embodiments, the homopolymeric nucleotide length is 2400 nucleotides. In some embodiments, the homopolymeric nucleotide length is 2500 nucleotides. In some embodiments, the homopolymeric nucleotide length is 2600 nucleotides. In some embodiments, the homopolymeric nucleotide length is 2700 nucleotides. In some embodiments, the homopolymeric nucleotide length is 2800 nucleotides. In some embodiments, the homopolymeric nucleotide length is 2900 nucleotides. In some embodiments, the homopolymeric nucleotide length is 3000 nucleotides. In some embodiments, the homopolymeric nucleotide length is 3100 nucleotides. In some embodiments, the homopolymeric nucleotide length is 3200 nucleotides. In some embodiments, the homopolymeric nucleotide length is 3300 nucleotides. In some embodiments, the homopolymeric nucleotide length is 3400 nucleotides. In some embodiments, the homopolymeric nucleotide length is 3500 nucleotides. In some embodiments, the homopolymeric nucleotide length is 3600 nucleotides. In some embodiments, the homopolymeric nucleotide length is 3700 nucleotides. In some embodiments, the homopolymeric nucleotide length is 3800 nucleotides. In some embodiments, the homopolymeric nucleotide length is 3900 nucleotides. In some embodiments, the homopolymeric nucleotide length is 4000 nucleotides. In some embodiments, the homopolymeric nucleotide length is 4100 nucleotides. In some embodiments, the homopolymeric nucleotide length is 4200 nucleotides. In some embodiments, the homopolymeric nucleotide length is 4300 nucleotides. In some embodiments, the homopolymeric nucleotide length is 4400 nucleotides. In some embodiments, the homopolymeric nucleotide length is 4500 nucleotides. In some embodiments, the homopolymeric nucleotide length is 4600 nucleotides. In some embodiments, the homopolymeric nucleotide length is 4700 nucleotides. In some embodiments, the homopolymeric nucleotide length is 4800 nucleotides. In some embodiments, the homopolymeric nucleotide length is 4900 nucleotides. In some embodiments, the homopolymeric nucleotide length is 5000 nucleotides.

In some embodiments, the homopolymeric nucleotide length is 50 or more nucleotides, 100 or more nucleotides, 150 or more nucleotides, or 200 or more nucleotides. In some embodiments, the homopolymeric nucleotide length is 50 or more nucleotides. In some embodiments, the homopolymeric nucleotide length is 100 or more nucleotides. In some embodiments, the homopolymeric nucleotide length is 150 or more nucleotides. In some embodiments, the homopolymeric nucleotide length is 200 or more nucleotides.

In some embodiments, the nucleotides comprising the homopolymeric nucleotides tract are selected from A, U, G, or C. In some embodiments, the nucleotides comprising the homopolymeric nucleotides are adenine. In some embodiments, the nucleotides comprising the homopolymeric nucleotides are uracil. In some embodiments, the nucleotides comprising the homopolymeric nucleotides are guanine. In some embodiments, the nucleotides comprising the homopolymeric nucleotides are cytosine.

In this application, the use of “or” means “and/or” unless stated otherwise. As used in this disclosure, the term “comprise” and variations of the term, such as “comprising” and “comprises,” are not intended to exclude other additives, components, integers or steps. As used in this application, the terms “about” and “approximately” are used as equivalents. Both terms are meant to cover any normal fluctuations appreciated by one of ordinary skill in the relevant art.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1A is a schematic that depicts binding of dyes and other ligands by different mechanisms to nucleic acids. In this schematic, an exemplary double-stranded DNA molecule is used to show dye-binding positions, for example, by an intercalating dye, a major groove binder, a minor groove binder or a bis-intercalator. FIG. 1B is a schematic that shows RNA secondary structures formed to which dyes can bind as depicted in FIG. 1A.

FIG. 2A depicts a gel showing RNase A analysis of EPO mRNA comprising poly A tails added cotranscriptionally, having poly A tail lengths of 25 nt, 50 nt and 114 nt for Sybr Gold™ dye-treated CE samples and traditional RNase A digested samples. FIG. 2B depicts a graph between observed tail length on the y-axis and theoretical tail length on the x-axis, for EPO mRNA having poly A tail lengths of 25 nt, 50 nt and 114 nt. The results are shown for Agilent™ dye-treated CE samples, Sybr Gold™ dye-treated CE samples, RNase A digested samples followed by Biorad MW analysis and theoretical tail lengths.

FIG. 3A depicts a capillary electrophoresis gel showing exemplary EPO mRNA having poly A tail lengths between 100-600 nucleotides after Sybr Gold™ binding followed by RNase A digestion. FIG. 3B depicts a capillary electrophoresis gel showing exemplary EPO mRNA having poly A tail lengths between 100-600 nucleotides after Agilent™ Intercalating Dye binding followed by RNase A digestion.

FIG. 4A depicts a gel showing traditional RNase A analysis of EPO mRNA having poly A tails of between 100-600 nucleotides in length. FIG. 4B depicts a graph between observed tail length on the y-axis and theoretical tail length on the x-axis, for EPO mRNA having poly A tail lengths of 100 nt, 200 nt, 300 nt, 400 nt, 500 nt and 600 nt. The results are shown for Agilent™ dye-treated CE samples, Sybr Gold™ dye-treated CE samples, RNase A digested samples followed by Biorad MW analysis and theoretical tail lengths.

FIG. 5A depicts capillary electropherograms following RNase A digestion of 1.2 μg of Sybr Gold™ dye-treated EPO mRNA samples having poly A tail lengths of 100, 200, 300, 400, 500 and 600 nt. FIG. 5B depicts capillary electropherograms following RNase A digestion of 1.2 μg of Agilent™ dye-treated EPO mRNA samples having poly A tail lengths of 100, 200, 300, 400, 500 and 600 nt.

FIG. 6A and FIG. 6B depict a comparison of signal intensity between Sybr Gold™ dye-treated and Agilent™ dye-treated EPO mRNA samples. FIG. 6A depicts capillary electropherogram following RNase A digestion of 1.2 μg of Sybr Gold™ dye-treated EPO mRNA having a poly A tail length of 200 nt. FIG. 6B depicts capillary electropherogram following RNase A digestion of 1.2 μg of Agilent™ dye-treated EPO mRNA having a poly A tail length of 200 nt.

FIG. 7 depicts a graph between observed tail length on the y-axis and theoretical tail length on the x-axis, for EPO mRNA having poly A tail lengths of between 100-1800 nucleotides, comparing RNaseA and RNase A/T1 digested products.

FIG. 8A depicts a gel following RNase A digestion of Sybr Gold™ dye-treated CFTR mRNA having a poly A tail length of 100, 200, 300, 400, 500 and 600 nt as compared to undigested controls. FIG. 8B depicts a gel following RNase A digestion of Agilent™ dye-treated CFTR mRNA having a poly A tail length of 100, 200, 300, 400, 500 and 600 nt as compared to undigested controls.

FIG. 9A depicts capillary electropherograms following RNase A digestion of 1.2 μg of Sybr Gold™ dye-treated CFTR mRNA samples having poly A tail lengths of 100, 200, 300, 400, 500 and 600 nt as compared to undigested controls. FIG. 9B depicts capillary electropherograms following RNase A digestion of 1.2 μg of Agilent™ dye-treated CFTR mRNA samples having poly A tail lengths of 100, 200, 300, 400, 500 and 600 nt as compared to undigested controls.

FIG. 10 depicts a graph between observed tail length on the y-axis and theoretical tail length on the x-axis, for CFTR mRNA having poly A tail lengths of 100 nt, 200 nt, 300 nt, 400 nt, 500 nt and 600 nt. The results are shown for Agilent™ dye-treated CE samples, Sybr Gold™ dye-treated CE samples, RNase A digested samples followed by Biorad MW analysis and theoretical tail lengths.

FIG. 11A depicts a gel following RNase A digestion of Sybr Gold™ dye-treated OTC mRNA having a poly A tail length of 100, 200, 300, 400, 500 and 600 nt as compared to undigested controls. FIG. 11B depicts a gel following RNase A digestion of Agilent™ dye-treated OTC mRNA having a poly A tail length of 100, 200, 300, 400, 500 and 600 nt as compared to undigested controls.

FIG. 12A depicts capillary electropherograms following RNase A digestion of 1.2 μg of Sybr Gold™ dye-treated OTC mRNA samples having poly A tail lengths of 100, 200, 300, 400, 500 and 600 nt as compared to undigested controls. FIG. 12B depicts capillary electropherograms following RNase A digestion of 1.2 μg of Agilent™ dye-treated OTC mRNA samples having poly A tail lengths of 100, 200, 300, 400, 500 and 600 nt as compared to undigested controls.

FIG. 13 depicts a graph between observed tail length on the y-axis and theoretical tail length on the x-axis, for OTC mRNA having poly A tail lengths of 100 nt, 200 nt, 300 nt, 400 nt, 500 nt and 600 nt. The results are shown for Agilent™ dye-treated CE samples, Sybr Gold™ dye-treated CE samples, RNase A digested samples followed by Biorad MW analysis and theoretical tail lengths.

FIG. 14A depicts a gel following RNase A digestion of Sybr Gold™ dye-treated MMA mRNA having a poly A tail length of 100, 200, 300, 400, 500 and 600 nt as compared to undigested controls. FIG. 14B depicts a gel following RNase A digestion of Agilent™ dye-treated MMA mRNA having a poly A tail length of 100, 200, 300, 400, 500 and 600 nt as compared to undigested controls.

FIG. 15A depicts capillary electropherograms following RNase A digestion of 1.2 μg of Sybr Gold™ dye-treated MMA mRNA samples having poly A tail lengths of 100, 200, 300, 400, 500 and 600 nt as compared to undigested controls. FIG. 15B depicts capillary electropherograms following RNase A digestion of 1.2 μg of Agilent™ dye-treated MMA mRNA samples having poly A tail lengths of 100, 200, 300, 400, 500 and 600 nt as compared to undigested controls.

FIG. 16 depicts a graph between observed tail length on the y-axis and theoretical tail length on the x-axis, for MMA mRNA having poly A tail lengths of 100 nt, 200 nt, 300 nt, 400 nt, 500 nt and 600 nt. The results are shown for Agilent™ dye-treated CE samples, Sybr Gold™ dye-treated CE samples, RNase A digested samples followed by Biorad MW analysis and theoretical tail lengths.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Batch: As used herein, the term “batch” refers to a quantity or amount of mRNA synthesized at one time, e.g., produced according to a single manufacturing order during the same cycle of manufacture. A batch may refer to an amount of mRNA synthesized in one reaction that occurs via a single aliquot of enzyme and/or a single aliquot of DNA template for continuous synthesis under one set of conditions. In some embodiments, a batch would include the mRNA produced from a reaction in which not all reagents and/or components are supplemented and/or replenished as the reaction progresses. The term “batch” would not mean mRNA synthesized at different times that are combined to achieve the desired amount.

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and their grammatical equivalents, are used interchangeably.

Full-length mRNA: As used herein, “full-length mRNA” is as characterized when using a specific assay, e.g., gel electrophoresis or detection using UV and UV absorption spectroscopy with separation by capillary electrophoresis. The length of an mRNA molecule that encodes a full-length polypeptide and as obtained following any of the purification methods described herein is at least 50% of the length of a full-length mRNA molecule that is transcribed from the target DNA, e.g., at least 60%, 70%, 80%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, 99.9% of the length of a full-length mRNA molecule that is transcribed from the target DNA and prior to purification according to any method described herein.

Functional: As used herein, a “functional” biological molecule is a biological molecule in a form in which it exhibits a property and/or activity by which it is characterized.

Homopolymer or homopolymeric nucleotides: As used herein, “homopolymer” or homopolymeric nucleotides,” and grammatical equivalents thereof, refers to a sequence of consecutive identical bases. In some embodiments, the nucleotides comprising the homopolymeric nucleotides are selected from A, U, G, or C. In some embodiments, the term “homopolymer” or “homopolymeric nucleotides,” refers to a sequence of substantially identical bases. For example, in some embodiments, the term includes a consecutive series of nucleotides that has one or more non-identical nucleotides.

Intercalating dyes: As used herein, intercalating dyes or ligands or agents bind between base pairs of a DNA double helix. Intercalating dyes are hydrophobic heterocyclic ring molecules that resemble the ring structure of base pairs, for example, ethidium bromide, acridine orange and actinomycin D. In some embodiments, the intercalating dye is an Agilent™ intercalating dye.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

Minor groove or Minor groove binding dye: As used herein, the “minor groove” refers to the narrower of two grooves in a DNA double helix where minor groove binding dyes bind by hydrogen bonding or hydrophobic interactions. In an RNA molecule, minor groove binding dyes bind non-covalently to secondary structure formed by single-stranded nucleic acid. In some embodiments, the minor groove binding dye is Sybr Gold™, a Hoechst dye, or 4′,6-diamidino-2-phenylindole (DAPI).

mRNA Integrity: As used herein, the term “mRNA integrity” generally refers to the quality of mRNA. In some embodiments, mRNA integrity refers to the percentage of mRNA that is not degraded after a purification process (e.g., a method described herein). mRNA integrity may be determined using methods particularly described herein, such as TAE Agarose gel electrophoresis or by SDS-PAGE with silver staining, or by methods well known in the art, for example, by RNA agarose gel electrophoresis (e.g., Ausubel et al., John Wiley & Sons, Inc., 1997, Current Protocols in Molecular Biology).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Pi stack: As used herein, pi stack refers to attractive, non-covalent interactions between aromatic rings, since they contain pi bonds. These interactions are important in nucleobase stacking within DNA and RNA molecules.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION

The present invention provides, among other things, a method of accurately measuring poly A tail length in an mRNA sample in a rapid and high-throughput manner. The present invention is based, in part, on the surprising and unexpected finding that binding of a minor groove binding dye with mRNA, followed by ribonuclease (RNase) digestion and capillary electrophoresis (CE) provides an accurate method of determining the poly A tail length of the mRNA. Capillary electrophoresis (CE) is typically carried out with intercalating dyes. In some embodiments, the intercalating dye is Agilent™ intercalating dye. The present invention is based, in part, on using minor groove binding dyes in capillary electrophoresis. In some embodiments, the minor groove binding dye is Sybr Gold™.

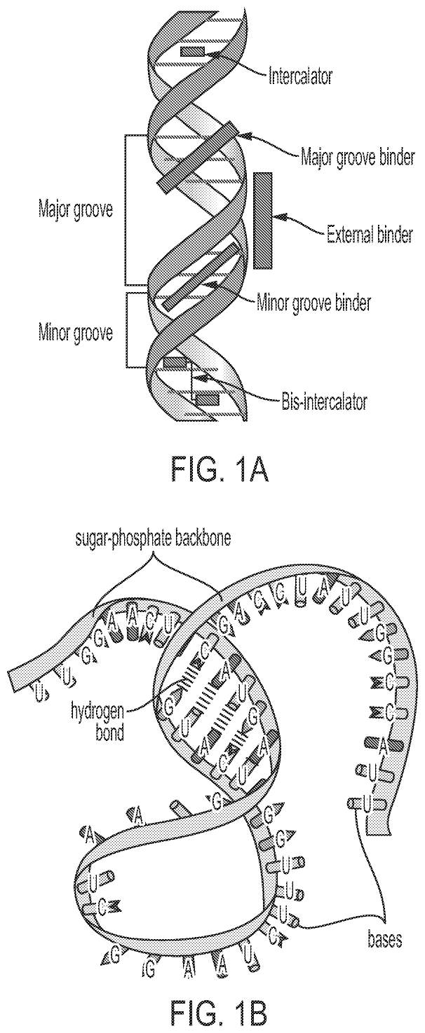

Poly A tails are helical; however, they are made of planar stacked bases. Without wishing to be bound by any particular theory, it is contemplated that minor groove binding dyes, including Sybr Gold™, can bind to the planar structure formed by poly A tails due to the interaction not being dependent on base stacking. Intercalating dyes such as Agilent™ intercalating dye produce a dampened poly A signal because intercalating dyes cannot properly pi-stack into the planar structure created by poly A tails. In contrast, Sybr Gold™ binds non-covalently via hydrogen bonding and hydrophobic signals to the helical structure of the poly A tail by binding to the minor grooves formed by the secondary structure of the single strand (FIG. 1B).

Various aspects of the invention are further described below.

Measuring mRNA Poly a Tail Length

As described in greater detail in the specification below, mRNAs may be synthesized according to any of a variety of known methods, including in vitro transcription (IVT). In some embodiments, the mRNA is capped and a poly A tail is added. In some embodiments, the poly A tail is added either post-transcriptionally or co-transcriptionally. The poly A tail confers stability to mRNA therapeutic product.

In some aspects, provided herein is a method of measuring poly A tail length in an mRNA sample, the method comprising: (a) contacting the mRNA sample with a minor-groove binding dye; (b) incubating the mRNA sample from (a) with one or more ribonucleases (RNase); and (c) assaying the sample from (b) by capillary electrophoresis (CE) to determine the poly A tail length of the mRNA. In some embodiments, one or more steps of the method is automated. In some embodiments, the method is high throughput.

### Minor Groove Binding Dyes

In some aspects, the method of this invention is based, in part, on binding of nucleic acids, including mRNA, to minor groove binding dyes (FIG. 1A and FIG. 1B). In some embodiments, the minor groove binding dye is Sybr Gold™, a Hoechst dye, or 4′,6-diamidino-2-phenylindole (DAPI). In some embodiments, the minor groove binding dye is Sybr Gold™. In some embodiments, the minor groove binding dye is a Hoechst dye. In some embodiments, the minor groove binding dye is 4′,6-diamidino-2-phenylindole (DAPI).

### Ribonucleases

In some embodiments, provided herein is a method, wherein the mRNA sample is incubated with one or more RNases selected from RNase A and RNase T1. In some embodiments, the one or more RNases is RNase A. In some embodiments, the one or more RNases is RNase T1. In some embodiments, the one or more RNases comprises RNase A1 and RNase T1. RNase A degrades RNA after C and U residues, while RNase T1 degrades after G residues. Digestion with RNase A and RNase T1 ensures that only poly A tails remain.

In some embodiments, the mRNA sample is incubated with one or more RNases is for about 15 minutes, 30 minutes, 45 minutes, or 60 minutes. In some embodiments, the mRNA sample is incubated with one or more RNases is for about 15 minutes. In some embodiments, the mRNA sample is incubated with one or more RNases is for about 30 minutes. In some embodiments, the mRNA sample is incubated with one or more RNases is for about 45 minutes. In some embodiments, the mRNA sample is incubated with one or more RNases is for about 60 minutes.

### Capillary Gel Electrophoresis

The method of this invention employs capillary electrophoresis coupled with a detection system for separation of mRNA based on equal mass-to-charge ratio of mRNA. The capillary gel electrophoresis method separates digestion products of various lengths from 25 nt to greater than about 5000 nucleotides. The length is accurately quantified based on standard size markers.

In some embodiments, the capillary electrophoresis is coupled with a fluorescence-based detection. For example, in some embodiments, the fluorescence-based detection method comprises laser-induced fluorescence detection.

In some embodiments, the capillary electrophoresis is coupled with UV absorption spectroscopy detection.

Synthesis of mRNA

mRNAs may be synthesized according to any of a variety of known methods. For example, mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

Synthesis of mRNA Using T3 RNA Polymerase

In some embodiments, mRNA is produced using T3 RNA Polymerase. T3 RNA Polymerase is a DNA-dependent RNA polymerase from the T3 bacteriophage that catalyzes the formation of RNA from DNA in the 5′→3′ direction on either single-stranded DNA or double-stranded DNA, and is able to incorporate modified nucleotide. T3 polymerase is extremely promoter-specific and transcribes only DNA downstream of a T3 promoter. T3 binds to a consensus promoter sequence of 5′-AATTAACCCTCACTAAAGGGAGA-3′ (SEQ ID NO: 1).

Synthesis of mRNA Using T7 RNA Polymerase

In some embodiments, mRNA is produced using T7 RNA Polymerase. T7 RNA Polymerase is a DNA-dependent RNA polymerase from the T7 bacteriophage that catalyzes the formation of RNA from DNA in the 5′→3′ direction. T7 polymerase is extremely promoter-specific and transcribes only DNA downstream of a T7 promoter. T7 binds to a consensus promoter sequence of 5′-TAATACGACTCACTATAGGGAGA-3′ (SEQ ID NO: 2). The T7 polymerase also requires a double stranded DNA template and Mg²⁺ ion as cofactor for the synthesis of RNA. It has a very low error rate.

Synthesis of mRNA Using SP6 RNA Polymerase

In some embodiments, mRNA is produced using SP6 RNA Polymerase. SP6 RNA Polymerase is a DNA-dependent RNA polymerase with high sequence specificity for SP6 promoter sequences. The SP6 polymerase catalyzes the 5′→3′ in vitro synthesis of RNA on either single-stranded DNA or double-stranded DNA downstream from its promoter; it incorporates native ribonucleotides and/or modified ribonucleotides and/or labeled ribonucleotides into the polymerized transcript. SP6 binds to a consensus promoter sequence of 5′-ATTTACGACACACTATAGAAGAA-3′ (SEQ ID NO: 3). Examples of such labeled ribonucleotides include biotin-, fluorescein-, digoxigenin-, aminoallyl-, and isotope-labeled nucleotides.

### DNA Template

Typically, a DNA template is either entirely double-stranded or mostly single-stranded with a suitable promoter sequence (e.g. T3, T7 or SP6 promoter).

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription, provided that they contain a double-stranded promoter upstream (and in the correct orientation) of the DNA sequence to be transcribed.

In some embodiments, the linearized DNA template has a blunt-end.

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature poly A sites, Shine-Dalgarno (SD) sequences, and/or other elements relevant to translation; and/or the DNA sequence may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which are described in US 20110081708, the contents of which are incorporated herein by reference in its entirety.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

Large-Scale mRNA Synthesis

In some embodiments, the mRNA with poly A tail can be synthesized in a large-scale. In some embodiments, mRNA is synthesized in at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more at a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount.

According to the present invention, 1-100 mg of RNA polymerase is typically used per gram (g) of mRNA produced. In some embodiments, about 1-90 mg, 1-80 mg, 1-60 mg, 1-50 mg, 1-40 mg, 10-100 mg, 10-80 mg, 10-60 mg, 10-50 mg of RNA polymerase is used per gram of mRNA produced. In some embodiments, about 5-20 mg of RNA polymerase is used to produce about 1 gram of mRNA. In some embodiments, about 0.5 to 2 grams of RNA polymerase is used to produce about 100 grams of mRNA. In some embodiments, about 5 to 20 grams of RNA polymerase is used to about 1 kilogram of mRNA. In some embodiments, at least 5 mg of RNA polymerase is used to produce at least 1 gram of mRNA. In some embodiments, at least 500 mg of RNA polymerase is used to produce at least 100 grams of mRNA. In some embodiments, at least 5 grams of RNA polymerase is used to produce at least 1 kilogram of mRNA. In some embodiments, about 10 mg, 20 mg, 30 mg, 40 mg, 50 mg, 60 mg, 70 mg, 80 mg, 90 mg, or 100 mg of plasmid DNA is used per gram of mRNA produced. In some embodiments, about 10-30 mg of plasmid DNA is used to produce about 1 gram of mRNA. In some embodiments, about 1 to 3 grams of plasmid DNA is used to produce about 100 grams of mRNA. In some embodiments, about 10 to 30 grams of plasmid DNA is used to about 1 kilogram of mRNA. In some embodiments, at least 10 mg of plasmid DNA is used to produce at least 1 gram of mRNA. In some embodiments, at least 1 gram of plasmid DNA is used to produce at least 100 grams of mRNA. In some embodiments, at least 10 grams of plasmid DNA is used to produce at least 1 kilogram of mRNA.

In some embodiments, the concentration of the RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the RNA polymerase is from about 10 to 50 nM, 20 to 50 nM, or 30 to 50 nM. A concentration of 100 to 10000 Units/ml of the RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

Linear or linearized DNA template (e.g., as described above and in an amount/concentration sufficient to provide a desired amount of RNA), the RNA polymerase reaction buffer, and RNA polymerase are combined to form the reaction mixture. The reaction mixture is incubated at between about 37° C. and about 42° C. for thirty minutes to six hours, e.g., about sixty to about ninety minutes.

In some embodiments, about 5 mM NTPs, about 0.05 mg/mL RNA polymerase, and about 0.1 mg/ml DNA template in a suitable RNA polymerase reaction buffer (final reaction mixture pH of about 7.5) is incubated at about 37° C. to about 42° C. for sixty to ninety minutes.

In some embodiments, a reaction mixture contains linearized double stranded DNA template with an RNA polymerase-specific promoter, RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° ° C. for 60 minutes. The polymerase reaction is then quenched by addition of DNase I and a DNase I buffer (when at 10× is 100 mM Tris-HCl, 5 mM MgCl₂ and 25 mM CaCl₂), pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and RNA polymerase at a concentration ranging from 0.01-0.1 mg/ml, e.g., the reaction mixture comprises NTPs at a concentration of 5 mM, the DNA template at a concentration of 0.1 mg/ml, and the RNA polymerase at a concentration of 0.05 mg/ml.

### Nucleotides

Various naturally-occurring or modified nucleosides may be used to product mRNA according to the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect mRNA from exonuclease degradation.

### 5′ Cap

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published US Application No. US 2016/0032356 and U.S. Provisional Application 62/464,327, filed Feb. 27, 2017, which are incorporated herein by reference.

### 3′-Poly A Tail

The presence of a “tail” at 3′ end serves to protect the mRNA from exonuclease degradation. The 3′ tail may be added before, after or at the same time of adding the 5′ Cap.

In some embodiments, the poly A tail is added co-transcriptionally. In some embodiments, the poly A tail is added post-transcriptionally. In some embodiments, the poly C tail is added co-transcriptionally. In some embodiments, the poly C tail is added post-transcriptionally.

In some embodiments, the poly A tail is 25-5,000 nucleotides in length. In some embodiments, the poly A tail 25 nucleotides in length. In some embodiments, the poly A tail 50 nucleotides in length. In some embodiments, the poly A tail 75 nucleotides in length. In some embodiments, the poly A tail 100 nucleotides in length. In some embodiments, the poly A tail 150 nucleotides in length. In some embodiments, the poly A tail 200 nucleotides in length. In some embodiments, the poly A tail 250 nucleotides in length. In some embodiments, the poly A tail 300 nucleotides in length. In some embodiments, the poly A tail 350 nucleotides in length. In some embodiments, the poly A tail 400 nucleotides in length. In some embodiments, the poly A tail 450 nucleotides in length. In some embodiments, the poly A tail 500 nucleotides in length. In some embodiments, the poly A tail 550 nucleotides in length. In some embodiments, the poly A tail 300 nucleotides in length. In some embodiments, the poly A tail 600 nucleotides in length. In some embodiments, the poly A tail 650 nucleotides in length. In some embodiments, the poly A tail 700 nucleotides in length. In some embodiments, the poly A tail 750 nucleotides in length. In some embodiments, the poly A tail 800 nucleotides in length. In some embodiments, the poly A tail 850 nucleotides in length. In some embodiments, the poly A tail 900 nucleotides in length. In some embodiments, the poly A tail 950 nucleotides in length. In some embodiments, the poly A tail 1000 nucleotides in length.

In some embodiments, the poly A tail 1500 nucleotides in length. In some embodiments, the poly A tail 2000 nucleotides in length. In some embodiments, the poly A tail 2500 nucleotides in length. In some embodiments, the poly A tail 3000 nucleotides in length. In some embodiments, the poly A tail 3500 nucleotides in length. In some embodiments, the poly A tail 4000 nucleotides in length. In some embodiments, the poly A tail 4500 nucleotides in length. In some embodiments, the poly A tail 5000 nucleotides in length.

Typically, a tail structure includes a poly A and/or poly C tail. (A, adenosine; C, cytosine). In some embodiments, a poly A or poly C tail on the 3′ terminus of mRNA includes at least 25 adenine or cytosine nucleotides, at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, at least 2 kb adenosine or cytosine nucleotides, at least 3 kb adenosine or cytosine nucleotides, at least 4 kb adenosine or cytosine nucleotides, at least 5 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly A and poly C tails with various lengths described herein. In some embodiments, a poly A tail structure includes at least 50%, 55%, 65%, 70%, 75%, 80%, 85%, 90%, 92%, 94%, 95%, 96%, 97%, 98%, or 99% adenosine nucleotides. In some embodiments, a poly A tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified. In other embodiments, the 5′ cap and/or the 3′ tail are added to the synthesized mRNA after the mRNA is purified.

Purification of mRNA

mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in US 2016/0040154, US 2015/0376220, PCT application PCT/US18/19954 entitled “METHODS FOR PURIFICATION OF MESSENGER RNA” filed on Feb. 27, 2018, and PCT application PCT/US18/19978 entitled “METHODS FOR PURIFICATION OF MESSENGER RNA” filed on Feb. 27, 2018, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of mRNA

Full-length or abortive transcripts of mRNA may be detected and quantified using any methods available in the art. In some embodiments, the synthesized mRNA molecules are detected using blotting, capillary electrophoresis, chromatography, fluorescence, gel electrophoresis, HPLC, silver stain, spectroscopy, ultraviolet (UV), or UPLC, or a combination thereof. Other detection methods known in the art are included in the present invention. In some embodiments, the synthesized mRNA molecules are detected using UV absorption spectroscopy with separation by capillary electrophoresis. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

In some embodiments, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than full length mRNA. The impurities include IVT contaminants, e.g., proteins, enzymes, free nucleotides and/or shortmers.

In some embodiments, mRNA produced according to the invention is substantially free of shortmers or abortive transcripts. In particular, mRNA produced according to the invention contains undetectable level of shortmers or abortive transcripts by capillary electrophoresis or Glyoxal gel electrophoresis. As used herein, the term “shortmers” or “abortive transcripts” refers to any transcripts that are less than full-length. In some embodiments, “shortmers” or “abortive transcripts” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail.

### Homopolymeric Nucleic Acid Tracts

In some aspects, the present invention provides a method of measuring homopolymeric nucleotide length in a nucleic acid, including mRNA, comprising contacting mRNA with a minor groove binding dye, subsequently treating the mRNA with one or more ribonucleases and performing capillary electrophoresis to determine the homopolymeric nucleotide length. In some embodiments, one or more steps of the method is automated. In some embodiments, the method is high throughput.

In some embodiments, the nucleotides comprising the homopolymeric nucleotides are selected from A, U, G, or C. In some embodiments, the nucleotides comprising the homopolymeric nucleotides are adenine. In some embodiments, the nucleotides comprising the homopolymeric nucleotides are uracil. In some embodiments, the nucleotides comprising the homopolymeric nucleotides are guanine. In some embodiments, the nucleotides comprising the homopolymeric nucleotides are cytosine.

The homopolymeric nucleotide length measured according to method of this invention ranges from about 25 nucleotides to greater than about 5000 nucleotides. In some embodiments, the homopolymeric nucleotide length is about 25 nucleotides, about 50 nucleotides, about 100 nucleotides, about 150 nucleotides, about 200 nucleotides or greater than about 200 nucleotides. In some embodiments, the homopolymeric nucleotide length is about 25 nucleotides, about 50 nucleotides, about 100 nucleotides, about 150 nucleotides, about 200 nucleotides or greater than about 200 nucleotides.

In some embodiments, the homopolymeric nucleotide length is between 50 nucleotides and 5,000 nucleotides. In some embodiments, the homopolymeric nucleotide length is 50 nucleotides. In some embodiments, the homopolymeric nucleotide length is 100 nucleotides. In some embodiments, the homopolymeric nucleotide length is 150 nucleotides. In some embodiments, the homopolymeric nucleotide length is 200 nucleotides. In some embodiments, the homopolymeric nucleotide length is 250 nucleotides. In some embodiments, the homopolymeric nucleotide length is 300 nucleotides. In some embodiments, the homopolymeric nucleotide length is 350 nucleotides. In some embodiments, the homopolymeric nucleotide length is 400 nucleotides. In some embodiments, the homopolymeric nucleotide length is 450 nucleotides. In some embodiments, the homopolymeric nucleotide length is 500 nucleotides. In some embodiments, the homopolymeric nucleotide length is 550 nucleotides. In some embodiments, the homopolymeric nucleotide length is 600 nucleotides. In some embodiments, the homopolymeric nucleotide length is 650 nucleotides. In some embodiments, the homopolymeric nucleotide length is 700 nucleotides. In some embodiments, the homopolymeric nucleotide length is 750 nucleotides. In some embodiments, the homopolymeric nucleotide length is 800 nucleotides. In some embodiments, the homopolymeric nucleotide length is 850 nucleotides. In some embodiments, the homopolymeric nucleotide length is 900 nucleotides. In some embodiments, the homopolymeric nucleotide length is 950 nucleotides. In some embodiments, the homopolymeric nucleotide length is 1000 nucleotides. In some embodiments, the homopolymeric nucleotide length is between about 1100 to 5000 nucleotides.

Homopolymeric nucleotides serve several functions, for example, protein binding regions, in upstream promoter elements, and in determining DNA location in a nucleosome structure. The method of the present invention provides, among other things, a method to measure the length of homopolymeric sequences in nucleic acids, including DNA and RNA.

In some aspects, the present invention further provides a method to measure lengths of homopolymers containing repeat units (SSRs), microsatellites, minisatellites and macrosatellites. SSRs are composed of 1-5 bp tandemly repeating units. For example, the most abundant SSRs are poly dA-poly dT and poly dG-poly dC, commonly found in non-coding regions and often greater than 9 bp in length. Poly dA-poly dT tracts are common in AT-rich sequences. The SSRs play a role in sequence specific DNA binding.

Microsatellites are composed of about 10 bp repeats, and are often found in regions including telomeres, comprising 6-8 bp repeats. In coding regions, frame-shift errors resulting from homopolymers lead to cancers. In several cancers, measuring the length of DNA microsatellites in a tumor sample reveals the measure of instability of microsatellites (whether they have grown shorter or longer), thus providing an indication of the progression of cancer.

In some embodiments, the method of the present invention is used to measure the length of minisatellites, which are composed of 10-100 bp repeating units. Minisatellites are often found in centromeres and heterochromatin regions. In some embodiments, the method of the present invention is used to measure the length of macrosatellites, which comprise greater than 100 bp of repeating units.

In some embodiments, the method of the present invention is used to measure the length of RNA homopolymers such as poly A and poly U that are used to make virus-like particles since they provide benefits over RNAs with normal composition comprising a mixture of bases.

In some embodiments, the method of the present invention is used to measure the length of homopolymers as quality control in next-generation sequencing reads comprising homopolymeric nucleotides. Repetitive DNA sequence assembly from short reads cannot determine the length of repetitive sequences such as microsatellites and these are often omitted from reported sequences.

In some embodiments, the method of the present invention is used to measure the length of homopolymeric nucleotides in tandem repeats, interspersed repeats, transposable elements, DNA transposons, retrotransposons, SINEs (Short Interspersed Nuclear Elements), LINEs (Long Interspersed Nuclear Elements), and CRISPR sequences.

### Examples

While certain compounds, compositions and methods of the present invention have been described with specificity in accordance with certain embodiments, the following examples serve only to illustrate the compounds of the invention and are not intended to limit the same.

Synthesis of mRNA

In each of the examples below, the synthesis of mRNA was conducted under complete RNase-free conditions. In the following examples, mRNA was synthesized via in vitro transcription from a linearized DNA template. To produce the desired mRNA precursor (IVT) construct, a mixture of about 8 mg of linearized DNA, INTPs (7.25 mM), DTT (10 mM), T7 RNA polymerase, RNase Inhibitor, Pyrophosphatase and reaction buffer (10×, 800 mM HEPES (pH 8.0), 20 mM Spermidine, 250 mM MgCl₂, pH 7.7) was prepared with RNase-free water to a final volume of 180 mL. The reaction mixture is incubated at 37° C. for a range of time between 20 minutes-60 minutes. Upon completion, the mixture is treated with DNase I for an additional 15 minutes and quenched accordingly.

### Addition of 5′ Cap and 3′ Tail

The purified mRNA product from the aforementioned IVT step was denatured at 65° C. for 10 minutes. Separately, portions of GTP (1.0 mM), S-adenosyl methionine, RNase inhibitor, 2′-O-Methyltransferase and guanylyl transferase are mixed together with reaction buffer (10×, 500 mM Tris-HCl (pH 8.0), 60 mM KCl, 12.5 mM MgCl₂) to a final concentration of 1.6 L. Upon denaturation, the mRNA was cooled on ice and then added to the reaction mixture. The combined solution was incubated for a range of time at 37º C. for 25-90 minutes. Upon completion, aliquots of ATP (2.0 mM), poly A polymerase and tailing reaction buffer (10×, 500 mM Tris-HCl (pH 8.0), 2.5 M NaCl, 100 mM MgCl₂) were added and the total reaction mixture was further incubated at 37° C. for a range of time from 20-45 minutes. Upon completion, the final reaction mixture was quenched and purified accordingly. Poly A tail length was measured according to the methods of the present invention, in some embodiments, as described in the following examples.

### Example 1. Measuring Poly a Tail Length of EPO mRNA Having a Short Tail Length of Less than 150 Nucleotides

This example illustrates a method of measuring poly A tail length in an exemplary EPO mRNA having exemplary tail lengths of about 25 nt, 50 nt and 114 nt.

Briefly, exemplary EPO mRNA samples were digested with one or more ribonucleases (RNases). In this example, in some embodiments, RNase A was used. The mRNA samples were then assayed by capillary electrophoresis to determine the poly A tail length of the mRNA.

The mRNA samples were incubated with organic solvent methanamide (formamide) at 75° C. for about 10 mins. The mRNA sample was then run using a Fragment Analyzer machine to perform CE. During this process, Sybr Gold dye was added to the RNA separation gel at a 1:10,000 dilution of a stock prior to adding the sample and starting the run. The dye was subsequently bound to the mRNA sample non-covalently via hydrogen bonding and hydrophobic interaction at room temperature.

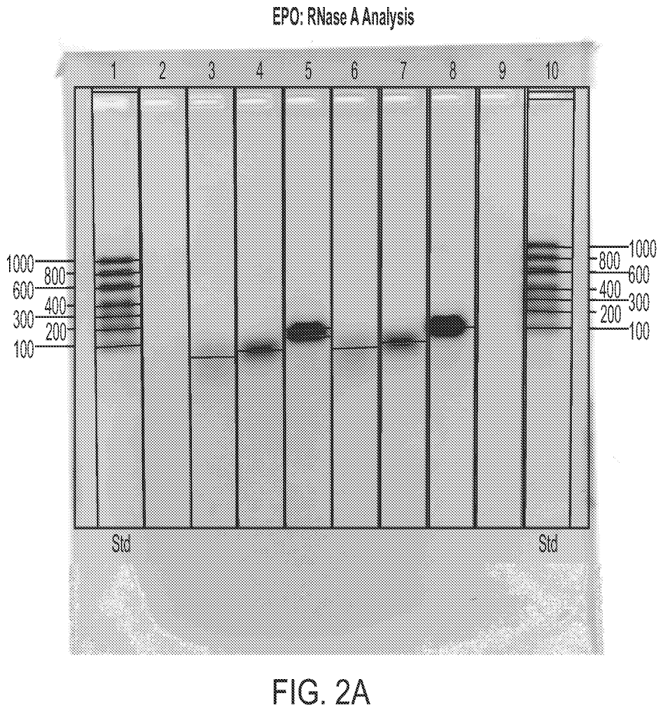

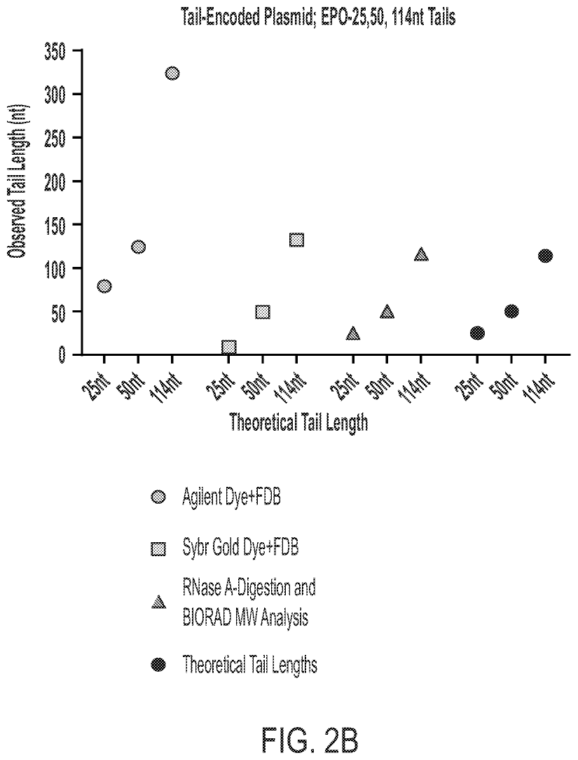

RNase A gel analysis of EPO mRNA comprising poly A tails added cotranscriptionally, having poly A tail lengths of 25 nt, 50 nt and 114 nt for Sybr Gold™ dye-treated CE samples and traditional RNase A digested samples is shown in FIG. 2A. The results from the analysis are depicted in a graph of observed tail length on the y-axis and theoretical tail length on the x-axis, for EPO mRNA having poly A tail lengths of 25 nt, 50 nt and 114 nt (FIG. 2B). The results are shown for Agilent™™ dye-treated samples (plotted from very low raw signals), Sybr Gold™ dye-treated samples, RNase A digested samples followed by Biorad MW analysis and theoretical tail lengths.

As seen from the results shown in FIG. 2B, the observed tail length is overestimated and highly inaccurate in the Agilent™™ dye-treated samples at all three tail lengths assayed, i.e. 25 nt, 50 nt and 114 nt. The Sybr Gold™ dye-treated CE samples and the RNase A digested samples followed by Biorad MW analysis showed good correlation between observed and theoretical tail lengths.

Overall, the results from this example showed that poly A tail lengths were accurately measured by a method wherein mRNA was bound with a minor groove-binding dye followed by RNase digestion and capillary electrophoresis. The method was highly accurate and observed poly A tail lengths were comparable to theoretical tail lengths. The method was comparable in accuracy to traditional RNase A digestion followed by Biorad MW analysis.

### Example 2. Measuring Poly a Tail Length of EPO mRNA Having a Long Tail Length of Greater than 100 Nucleotides

This example illustrates a method of measuring poly A tail length in an exemplary EPO mRNA having exemplary tail lengths of about 100 nt, 200 nt, 300 nt, 4000 nt, 500 nt and 600 nt.

Briefly, exemplary EPO mRNA samples were digested with one or more ribonucleases (RNases). In this example, in some embodiments, RNase A was used. The mRNA samples were then assayed by capillary electrophoresis to determine the poly A tail length of the mRNA.

The mRNA samples were incubated with organic solvent methanamide (formamide) at 75° ° C. for about 10 mins. The mRNA sample was then run using a Fragment Analyzer machine to perform CE. During this process, Sybr Gold dye was added to the RNA separation gel at a 1:10,000 dilution of a stock prior to adding the sample and starting the run. The dye was subsequently bound to the mRNA sample non-covalently via hydrogen bonding and hydrophobic interaction at room temperature.

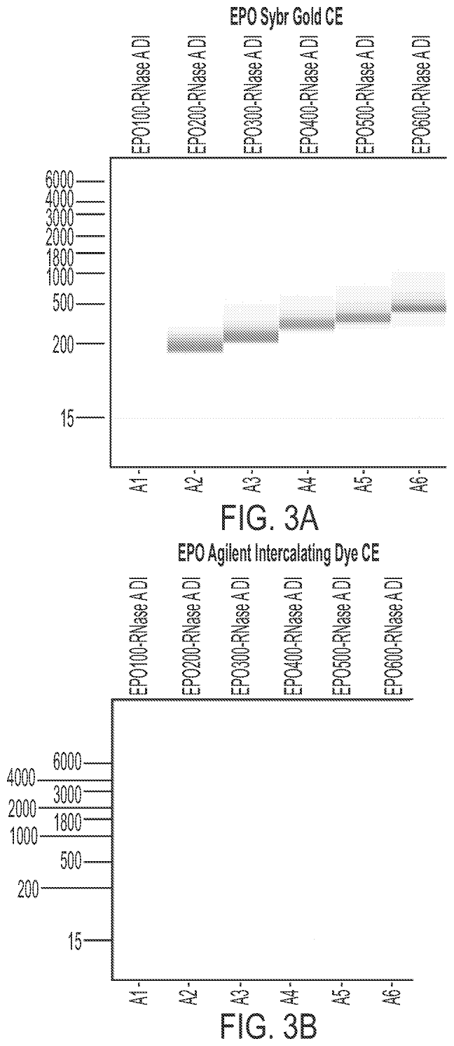

RNase A gel analysis of EPO mRNA comprising poly A tails having poly A tail lengths of 100 nt, 200 nt, 300 nt, 400 nt, 500 nt and 600 nt for Sybr Gold™ dye-treated CE is shown in FIG. 3A. RNase A gel analysis of EPO mRNA comprising poly A tails having poly A tail lengths of 100 nt, 200 nt, 300 nt, 400 nt, 500 nt and 600 nt for Agilent™ intercalating dye-treated CE is shown in FIG. 3B.

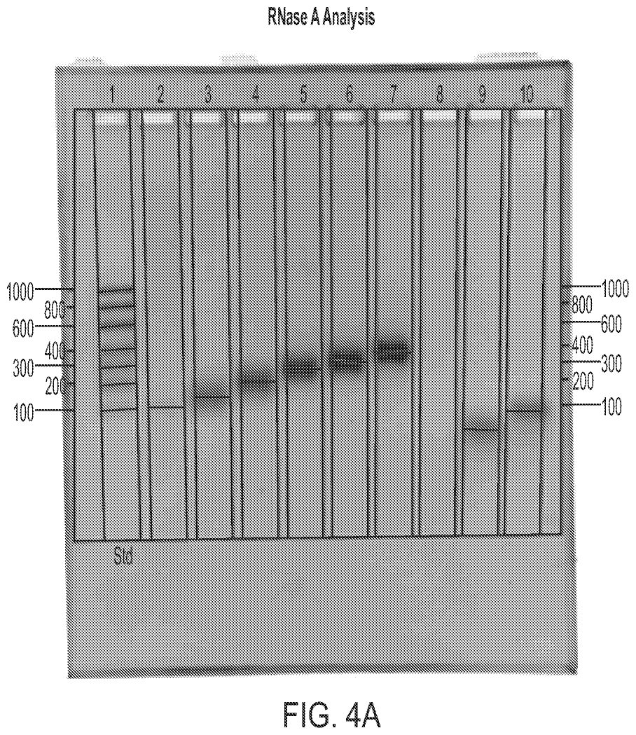

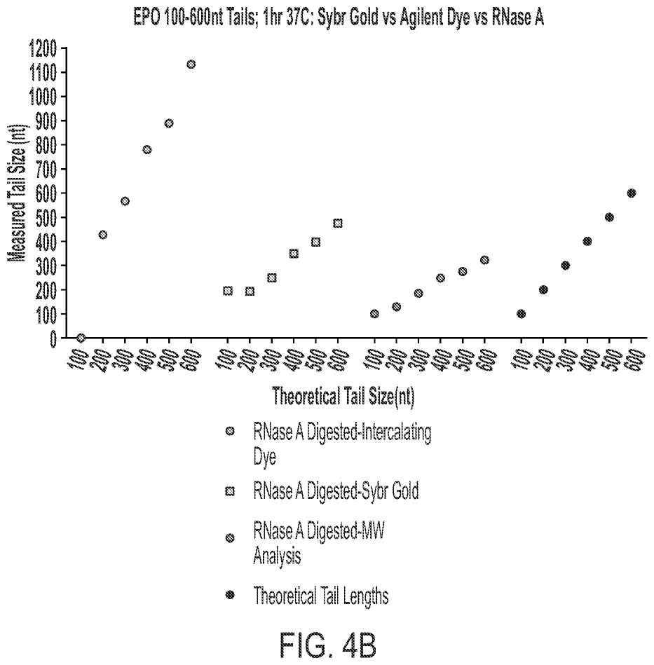

In some embodiments, poly A tail lengths were also assessed by the traditional RNase A method. Briefly, mRNA samples were digested using RNase A for 30 minutes, and run on 2% agarose gels for 2 hours and 30 minutes. The products are shown in FIG. 4A. Poly A tail lengths were then analysed by Biorad MW analysis.

The results from the analysis are shown in a graph of observed tail length on the y-axis and theoretical tail length on the x-axis, for EPO mRNA having poly A tail lengths of 100 nt, 200 nt, 300 nt, 400 nt, 500 nt and 600 nt (FIG. 4B). The results are shown for Agilent™ dye-treated samples (plotted from very low raw signals), Sybr Gold™ dye-treated samples, RNase A digested samples followed by Biorad MW analysis and theoretical tail lengths.

As seen from the results shown in FIG. 4B, the observed tail length is highly inaccurate in the Agilent™ dye-treated samples at all six tail lengths assayed, i.e. 100 nt, 200 nt, 300 nt, 400 nt, 500 nt and 600 nt. The observed tail lengths are highly underestimated for 100 nt and overestimated for 200-600 nt tail lengths. Sybr Gold™ dye-treated CE samples showed good correlation between observed and theoretical tail lengths. The Sybr Gold™-treated CE samples showed improved correlation between observed and theoretical tail lengths than the traditional RNase A method followed by Biorad MW analysis.

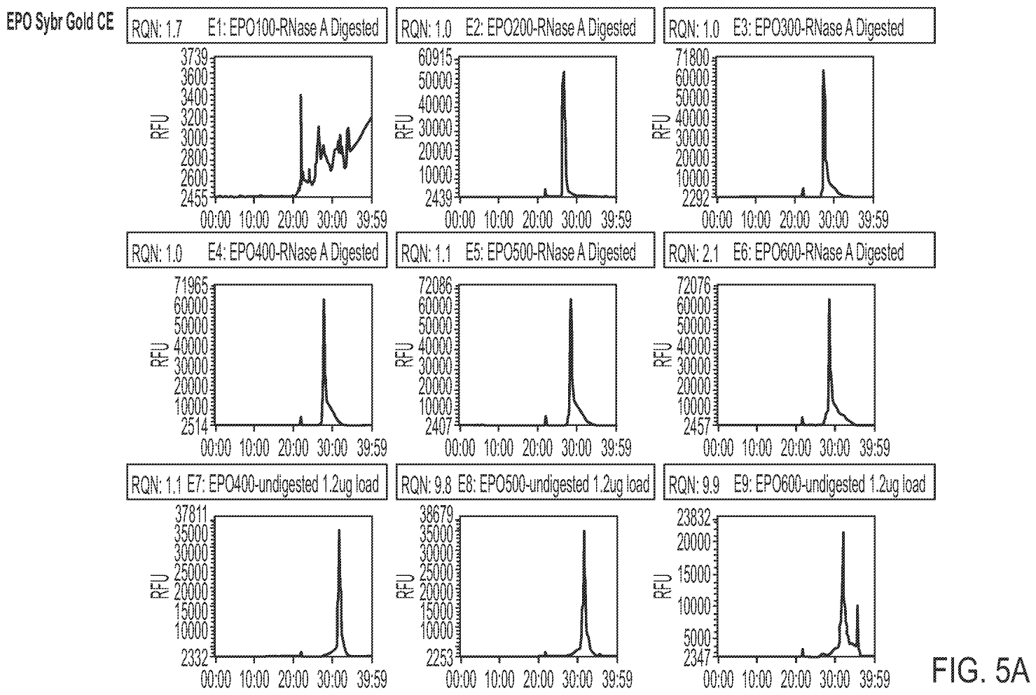

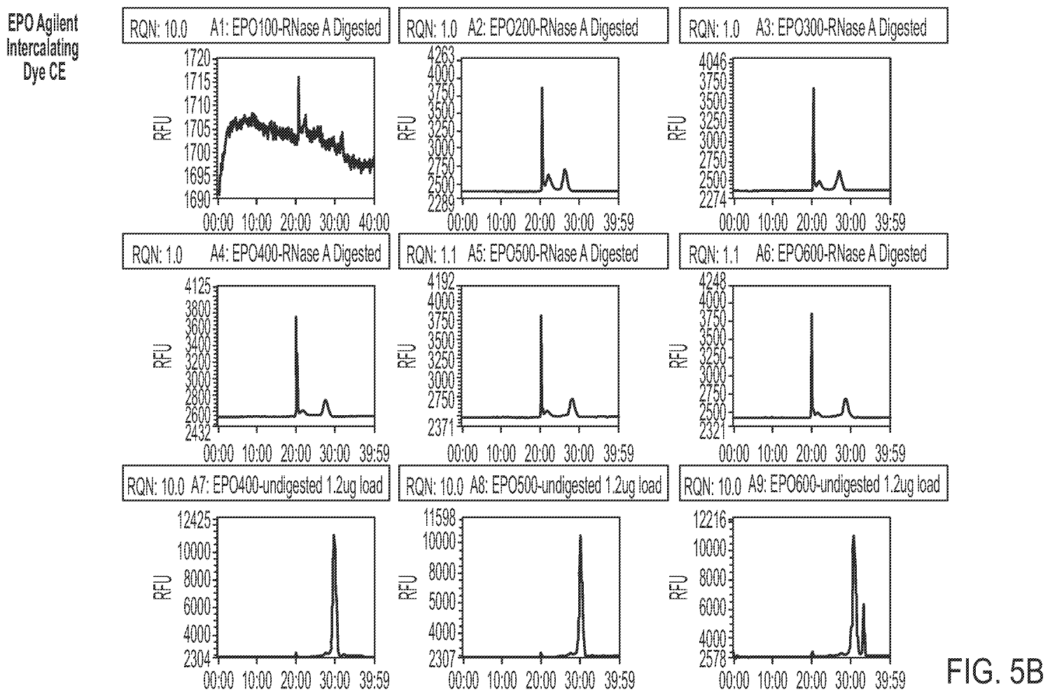

Capillary electropherogram following RNase A digestion of 1.2 μg of Sybr Gold™ dye-treated EPO mRNA samples having poly A tail lengths of 100, 200, 300, 400, 500 and 600 nt is depicted in FIG. 5A. Capillary electropherograms following RNase A digestion of 1.2 μg of Agilent™ dye-treated EPO mRNA samples having poly A tail lengths of 100, 200, 300, 400, 500 and 600 nt is depicted in FIG. 5B.

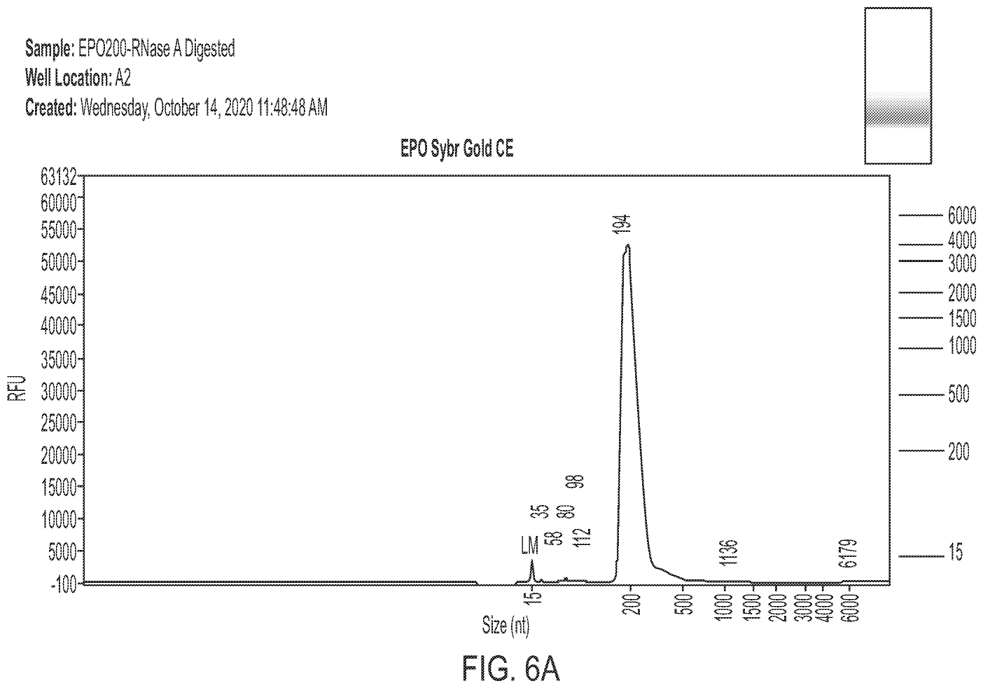

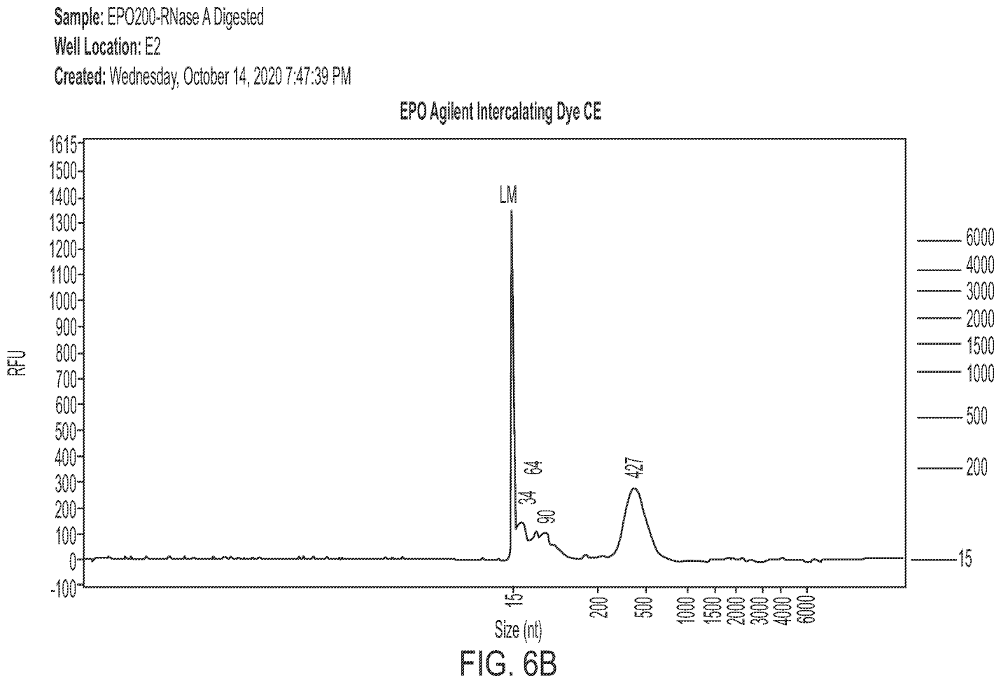

A comparison of signal intensity between Sybr Gold™ dye-treated and Agilent™ dye-treated EPO mRNA samples is seen FIGS. 6A and 6B. A capillary electropherogram following RNase A digestion of 1.2 μg of Sybr Gold™ dye-treated EPO mRNA having a poly A tail length of 200 nt is shown in FIG. 6A. A capillary electropherogram following RNase A digestion of 1.2 μg of Agilent™ dye-treated EPO mRNA having a poly A tail length of 200 nt is shown in FIG. 6B.

Overall, the results from this example showed that poly A tail lengths were accurately measured by a method wherein mRNA was bound with a minor groove-binding dye followed by RNase digestion and capillary electrophoresis. The method was highly accurate and observed poly A tail lengths were comparable to theoretical tail lengths. The method was comparable in accuracy to traditional RNase A digestion followed by Biorad MW analysis.

### Example 3. Comparison of the Accuracy of Poly a Tail Length Measurements by a Method Comprising mRNA Digestion Using Either RNase a Alone or Both RNase a and RNase T1 Enzymes

This example illustrates a comparison of the accuracy of poly A tail length measurements using a method comprising mRNA digestion with RNase A alone or both RNase A and RNase T1 enzymes.

RNase A degrades RNA after C and U residues, while RNase T1 degrades after G residues. Digestion with RNase A and RNase T1 ensures that only poly A tails remain. In this example, accuracy of poly A tail length measurement was compared for exemplary EPO mRNA samples having poly A tail lengths ranging from 50-1800 nucleotides long, digested with RNase A or RNase A and RNase T1.

In this example, samples of EPO mRNAs having theoretical poly A tail lengths between 50 and 1800 nucleotides (for example, 50 nt, 100 nt, 200 nt, 500 nt, 1000 nt, 1500 nt and 1800 nt) were digested with RNase A for 30 minutes. The digestion products were run on a 2% agarose gel and molecular weight was evaluated on BIORAD to determine poly A tail lengths. A graph was plotted between observed tail length on the y-axis and theoretical tail length on the x-axis.

In parallel, samples of EPO mRNAs having poly A tail lengths between 50 and 1800 nucleotides (for example, 50 nt, 100 nt, 200 nt, 500 nt, 1000 nt, 1500 nt and 1800 nt) were digested with RNase A and RNase T1 for 30 minutes. The digestion products were run on a 2% agarose gel and molecular weight was evaluated on BIORAD to determine poly A tail lengths. A graph was plotted between observed tail length on the y-axis and theoretical tail length on the x-axis.

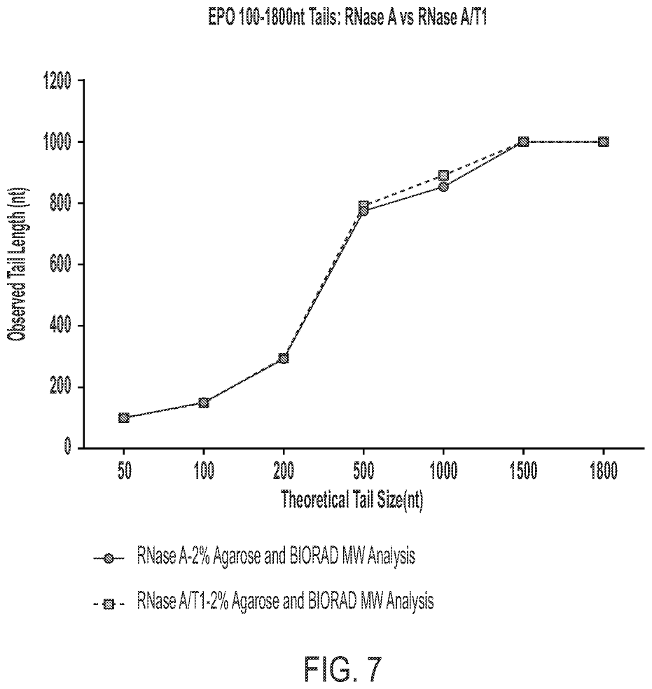

The results are shown in FIG. 7 and Table 1. The graph demonstrated correlation between observed and theoretical tail lengths. The observed tail lengths approached theoretical tail lengths.

TABLE 1
Ratio of Theoretical vs. Observed Tail Length
after RNase A or RNase A/T1 digestion.

| | | | Sybr Gold | |
|---|---|---|---|---|
| | Sybr Gold | Ratio of | RNAse | Ratio of |
| | RNAse A | Observed: | A/T1 | Observed: |
| Theoretical | (Observed | Theoretical | (Observed | Theoretical |
| Tail Length | Tail Length) | Tail Length | Tail Length) | Tail Length |
| 50 | 178.5 | 3.57 | 244 | 4.88 |
| 100 | 232 | 2.32 | 278.5 | 2.78 |
| 200 | 379.5 | 1.89 | 401 | 2.00 |
| 500 | 1386.5 | 2.77 | 1315 | 2.63 |
| 1000 | 2608.5 | 2.60 | 2237 | 2.24 |
| 1500 | 0 | 0 | 0 | 0 |
| 1800 | 7697 | 4.27 | 0 | 0 |

This example also demonstrated that observed tail lengths were comparable in RNase A and RNase A/T1 digested EPO mRNA samples having tail lengths between 50-1800 nucleotides.

### Example 4. Method of Measuring Poly a Tail Length of CFTR mRNA Having a Long Tail Length of Greater than 100 Nucleotides

This example illustrates a method of measuring poly A tail length in an exemplary CFTR mRNA having exemplary tail lengths of about 100 nt, 200 nt, 300 nt, 4000 nt, 500 nt and 600 nt.

Briefly, exemplary CFTR mRNA samples were digested with one or more ribonucleases (RNases). In this example, in some embodiments, RNase A was used. The mRNA samples were then assayed by capillary electrophoresis to determine the poly A tail length of the mRNA.

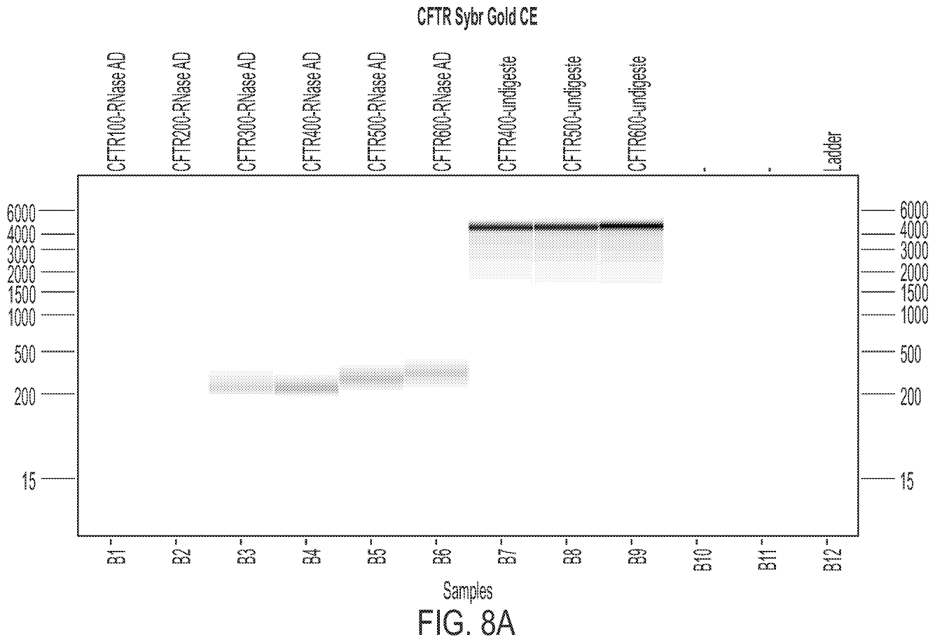

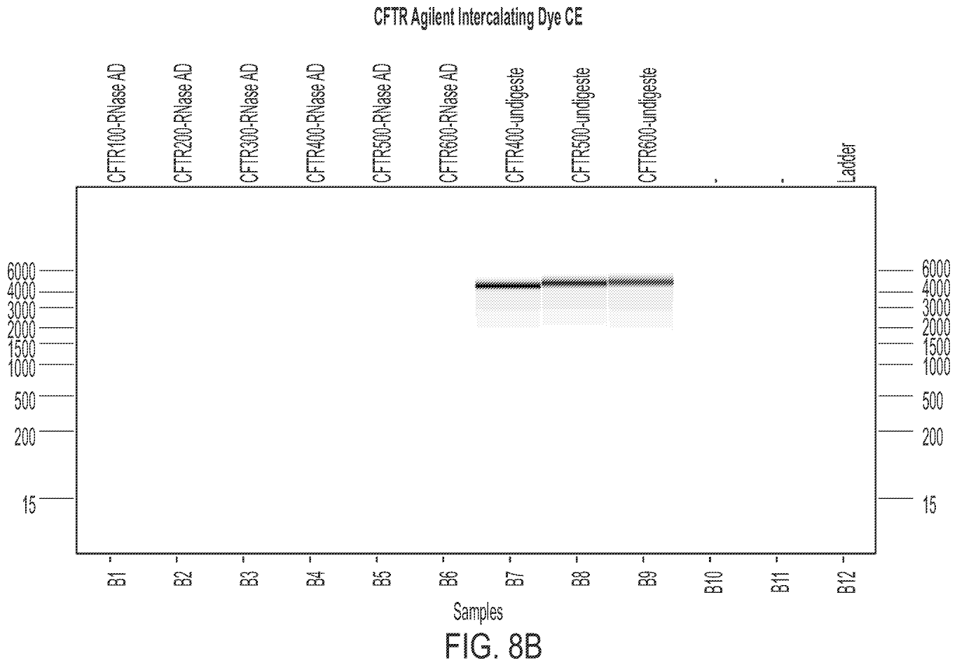

The mRNA samples were incubated with organic solvent methanamide (formamide) at 75° ° C. for about 10 mins. The mRNA sample was then run using a Fragment Analyzer machine to perform CE. During this process, Sybr Gold dye was added to the RNA separation gel at a 1:10,000 dilution of a stock prior to adding the sample and starting the run. The dye was subsequently bound to the mRNA sample non-covalently via hydrogen bonding and hydrophobic interaction at room temperature. RNase A gel analysis of CFTR mRNA comprising poly A tails having poly A tail lengths of 100 nt, 200 nt, 300 nt, 400 nt, 500 nt and 600 nt for Sybr Gold™ dye-treated samples relative to undigested controls having poly A tail lengths of 400 nt, 500 nt and 600 nt is shown in FIG. 8A. RNase A gel analysis of CFTR mRNA comprising poly A tails having poly A tail lengths of 100 nt, 200 nt, 300 nt, 400 nt, 500 nt and 600 nt for Agilent™ intercalating dye-treated CE relative to undigested controls having poly A tail lengths of 400 nt, 500 nt and 600 nt is shown in FIG. 8B.

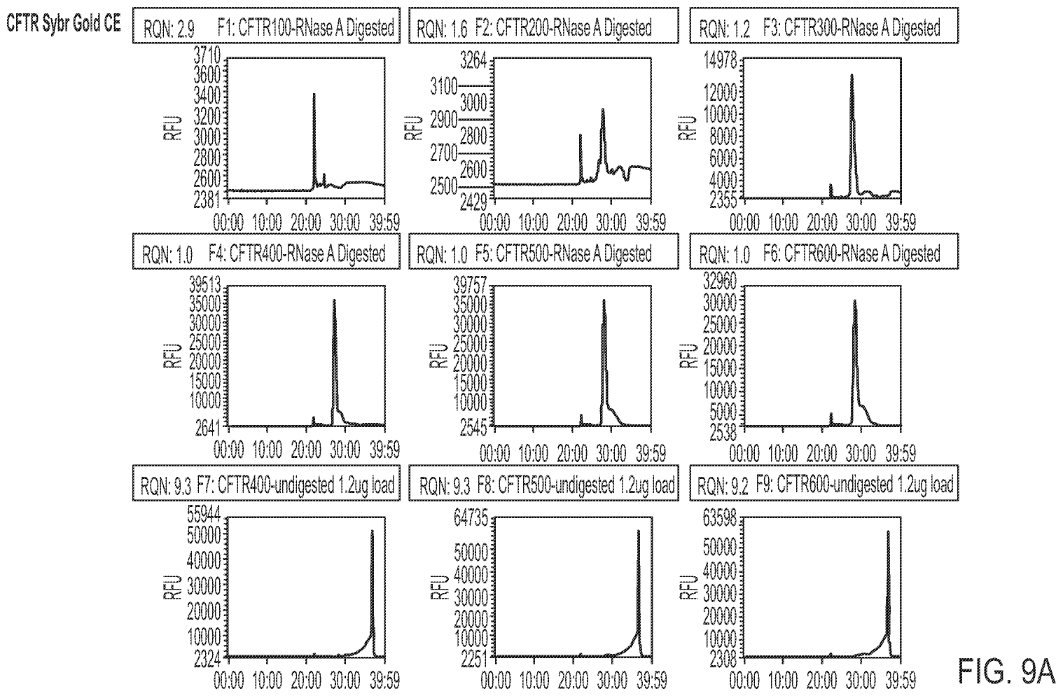

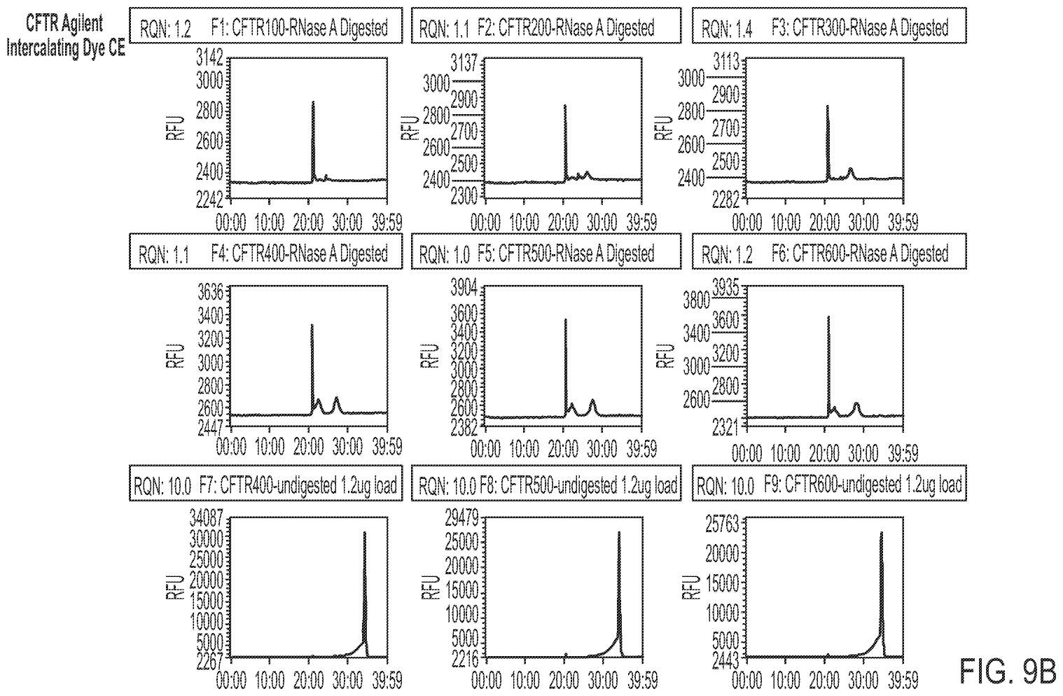

Capillary electropherograms depicting peaks for Sybr Gold™ dye-treated CFTR mRNA samples comprising poly A tail lengths of 100 nt, 200 nt, 300 nt, 400 nt, 500 nt and 600 nt relative to undigested controls having poly A tail lengths of 400 nt, 500 nt and 600 nt are shown in FIG. 9A. Capillary electropherograms depicting peaks for Agilent™ intercalating dye-treated CFTR mRNA samples comprising poly A tail lengths of 100 nt, 200 nt, 300 nt, 400 nt, 500 nt and 600 nt relative to undigested controls having poly A tail lengths of 400 nt, 500 nt and 600 nt are shown in FIG. 9B. The results show that the signal strength is much lower in the Agilent™ dye-treated samples (FIG. 9B) relative to Sybr Gold™-treated samples (FIG. 9A).

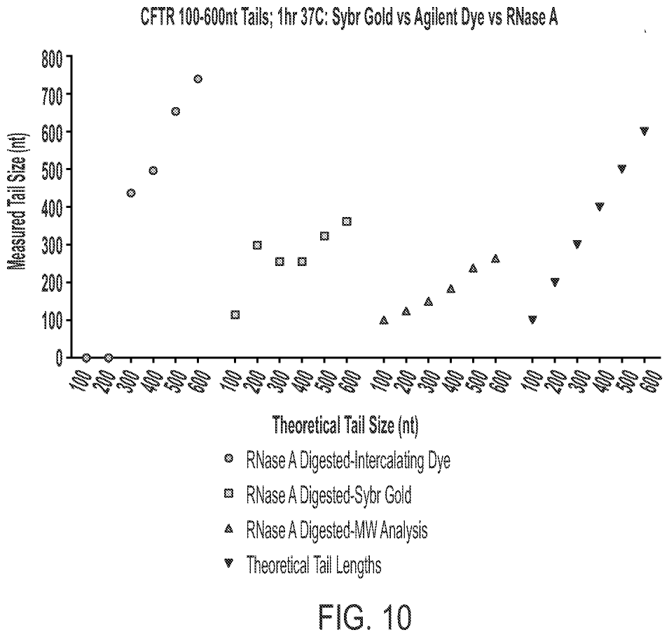

The results from the analysis are shown in a graph of observed tail length on the y-axis and theoretical tail length on the x-axis, for CFTR mRNA having poly A tail lengths of 100 nt, 200 nt, 300 nt, 400 nt, 500 nt and 600 nt (FIG. 10). The results are shown for Agilent™ dye-treated samples (plotted from very low raw signals), Sybr Gold™ dye-treated samples, RNase A digested samples followed by Biorad MW analysis and theoretical tail lengths.

As seen in FIG. 10, the observed tail length is underestimated at 100 nt and 200 nt lengths but overestimated at tail lengths between 300-600 nt in the Agilent™ dye-treated samples, overall resulting in inaccurate poly A tail length measurements.

RNase A digested samples followed by Biorad MW analysis showed accurate tail length measurements comparable to theoretical tail lengths for short tail lengths such as 100 nt. However, between 200-600 nt lengths, the observed tail lengths were underestimated relative to theoretical tail lengths.

The Sybr Gold™-treated samples showed accurate short tail length measurements at 100 nt. Longer tail lengths were measured more accurately than observed with the RNase A method followed by Biorad MW measurements for tail lengths between 200-600 nt.

### Example 5. Method of Measuring Poly a Tail Length of OTC mRNA Having a Long Tail Length of Greater than 100 Nucleotides

This example illustrates a method of measuring poly A tail length in an exemplary OTC mRNA having exemplary tail lengths of about 100 nt, 200 nt, 300 nt, 400 nt, 500 nt and 600 nt.

Briefly, exemplary OTC mRNA samples were digested with one or more ribonucleases (RNases). In this example, in some, RNase A was used. The mRNA samples were then assayed by capillary electrophoresis to determine the poly A tail length of the mRNA.

The mRNA samples were incubated with organic solvent methanamide (formamide) at 75° C. for about 10 mins. The mRNA sample was then run using a Fragment Analyzer machine to perform CE. During this process, Sybr Gold dye was added to the RNA separation gel at a 1:10,000 dilution of a stock prior to adding the sample and starting the run. The dye was subsequently bound to the mRNA sample non-covalently via hydrogen bonding and hydrophobic interaction at room temperature.

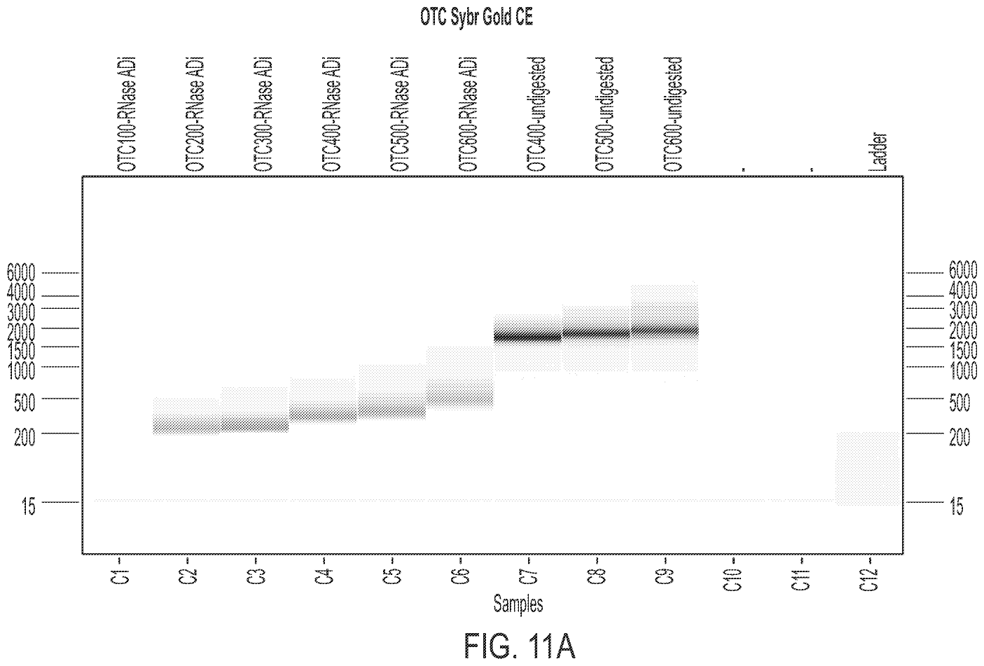

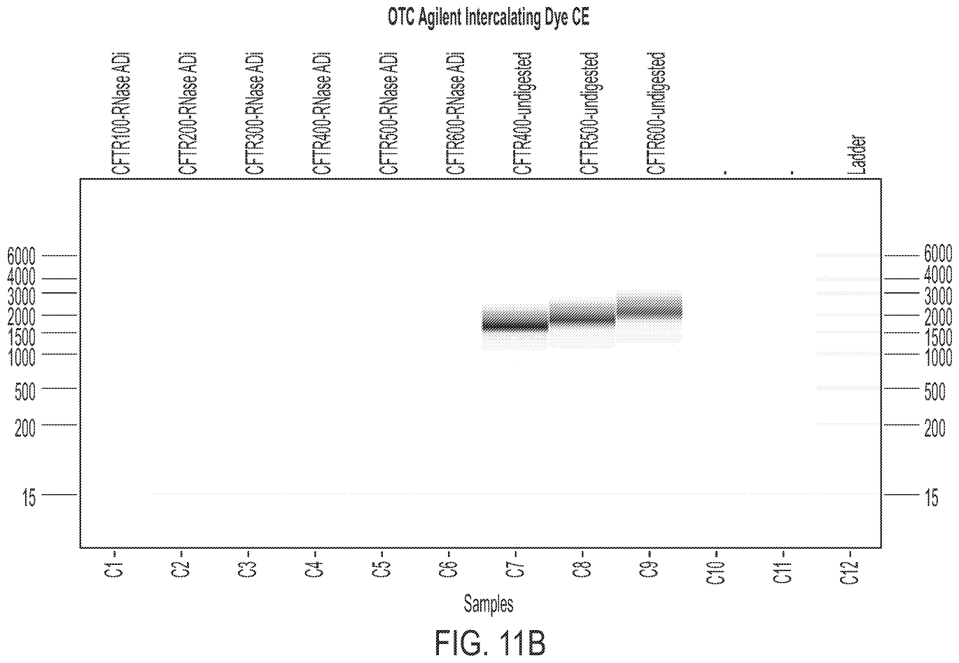

RNase A gel analysis of OTC mRNA comprising poly A tails having poly A tail lengths of 100 nt, 200 nt, 300 nt, 400 nt, 500 nt and 600 nt for Sybr Gold™ dye-treated samples relative to undigested controls having poly A tail lengths of 400 nt, 500 nt and 600 nt is shown in FIG. 11A. RNase A gel analysis of OTC mRNA comprising poly A tails having poly A tail lengths of 100 nt, 200 nt, 300 nt, 400 nt, 500 nt and 600 nt for Agilent™ intercalating dye-treated CE relative to undigested controls having poly A tail lengths of 400 nt, 500 nt and 600 nt is shown in FIG. 11B.

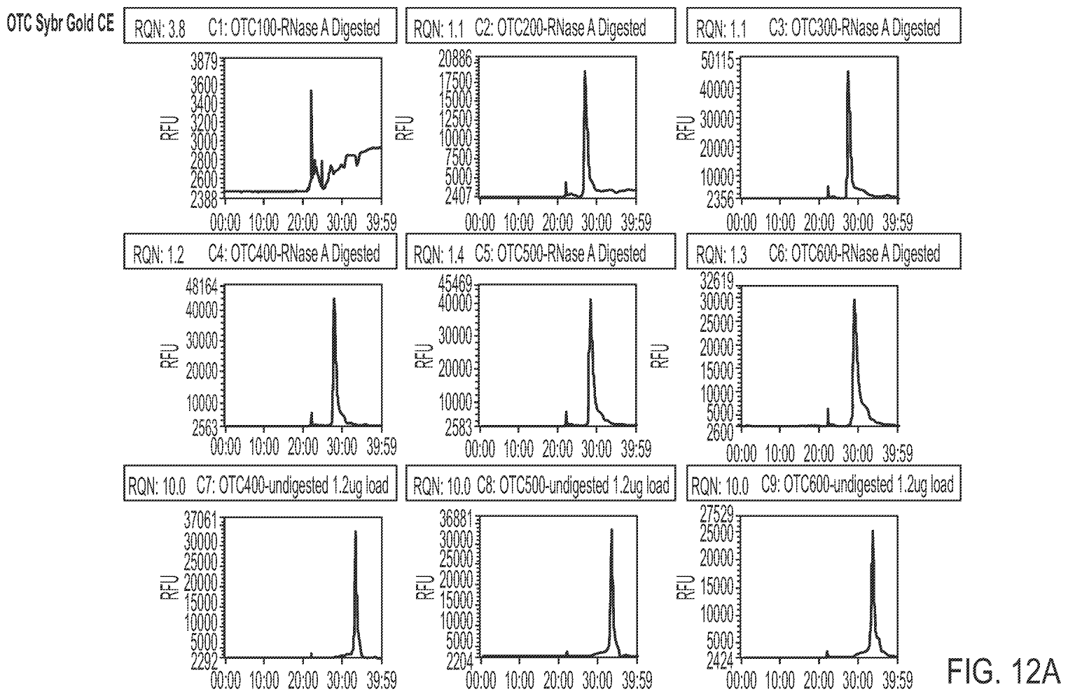

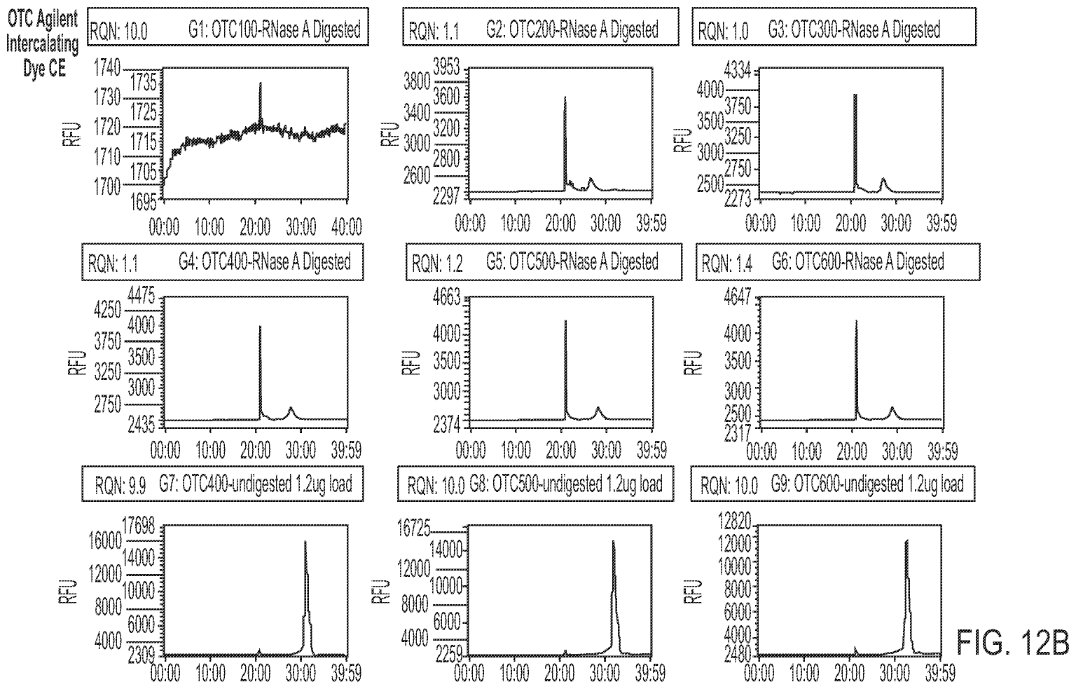

Capillary electropherograms depicting peaks for Sybr Gold™ dye-treated OTC mRNA samples comprising poly A tail lengths of 100 nt, 200 nt, 300 nt, 400 nt, 500 nt and 600 nt relative to undigested controls having poly A tail lengths of 400 nt, 500 nt and 600 nt are shown in FIG. 12A. Capillary electropherograms depicting peaks for Agilent™ intercalating dye-treated OTC mRNA samples comprising poly A tail lengths of 100 nt, 200 nt, 300 nt, 400 nt, 500 nt and 600 nt relative to undigested controls having poly A tail lengths of 400 nt, 500 nt and 600 nt are shown in FIG. 12B. The results show that the signal strength is much lower in the Agilent™ dye-treated samples (FIG. 12B) relative to Sybr Gold™-treated samples (FIG. 12A).

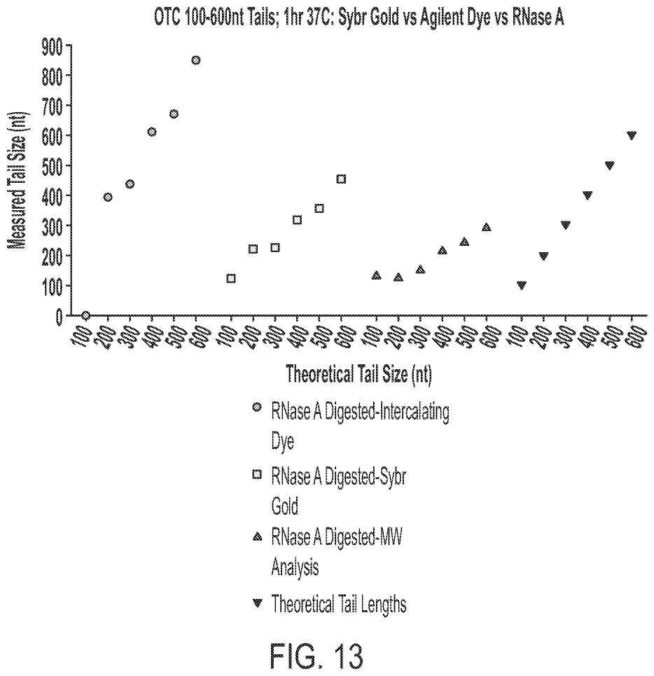

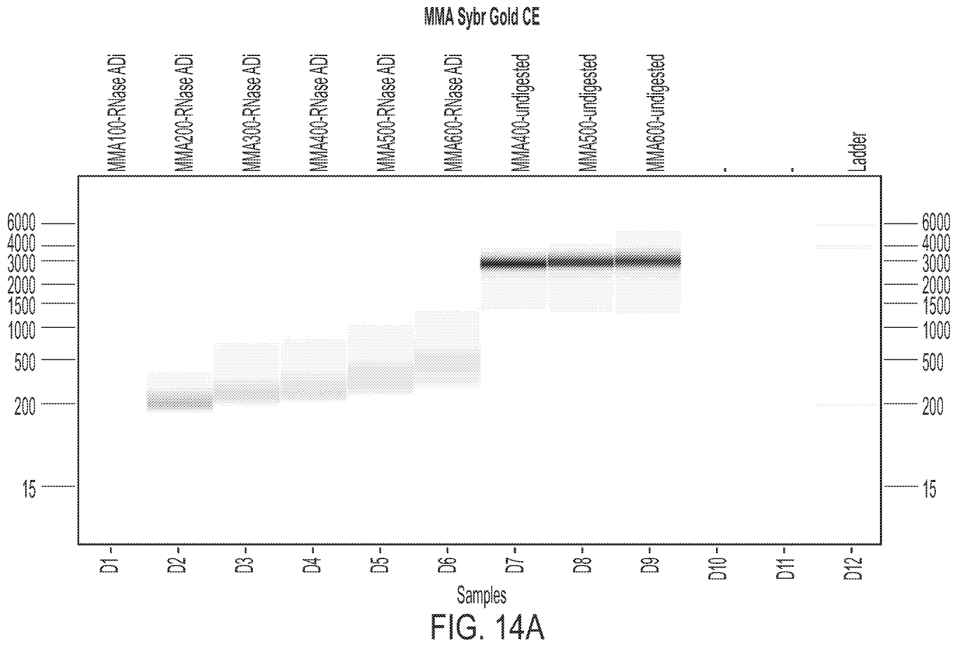

The results from the analysis are shown in a graph of observed tail length on the y-axis and theoretical tail length on the x-axis, for OTC mRNA having poly A tail lengths of 100 nt, 200 nt, 300 nt, 400 nt, 500 nt and 600 nt (FIG. 13). The results are shown for Agilent™ dye-treated samples (plotted from very low raw signals), Sybr Gold™ dye-treated samples, RNase A digested samples followed by Biorad MW analysis and theoretical tail lengths.

As seen in FIG. 13, the observed tail length is underestimated at 100 nt length but overestimated at tail lengths between 200-600 nt in the Agilent™ dye-treated samples, overall resulting in inaccurate poly A tail length measurements.

RNase A digested samples followed by Biorad MW analysis showed accurate tail length measurements comparable to theoretical tail lengths for short tail lengths such as 100 nt. However, between 200-600 nt tail lengths, the observed tail lengths were underestimated relative to theoretical tail lengths.

The Sybr Gold™-treated samples showed accurate tail length measurements between 100 nt-400 nt. Longer tail lengths such as 500 nt and 600 nt corresponded more closely to theoretical tail lengths than observed with the RNase A method followed by Biorad MW measurements for tail lengths between 100-600 nt.

### Example 6. Method of Measuring Poly a Tail Length of MMA mRNA Having a Long Tail Length of Greater than 100 Nucleotides

This example illustrates a method of measuring poly A tail length in an exemplary MMA mRNA having exemplary tail lengths of about 100 nt, 200 nt, 300 nt, 4000 nt, 500 nt and 600 nt.

Briefly, exemplary MMA mRNA samples were digested with one or more ribonucleases (RNases). In this example, in some embodiments, RNase A was used. The mRNA samples were then assayed by capillary electrophoresis to determine the poly A tail length of the mRNA.

The mRNA samples were incubated with organic solvent methanamide (formamide) at 75° C. for about 10 mins. The mRNA sample was then run using a Fragment Analyzer machine to perform CE. During this process, Sybr Gold dye was added to the RNA separation gel at a 1:10,000 dilution of a stock prior to adding the sample and starting the run. The dye was subsequently bound to the mRNA sample non-covalently via hydrogen bonding and hydrophobic interaction at room temperature.

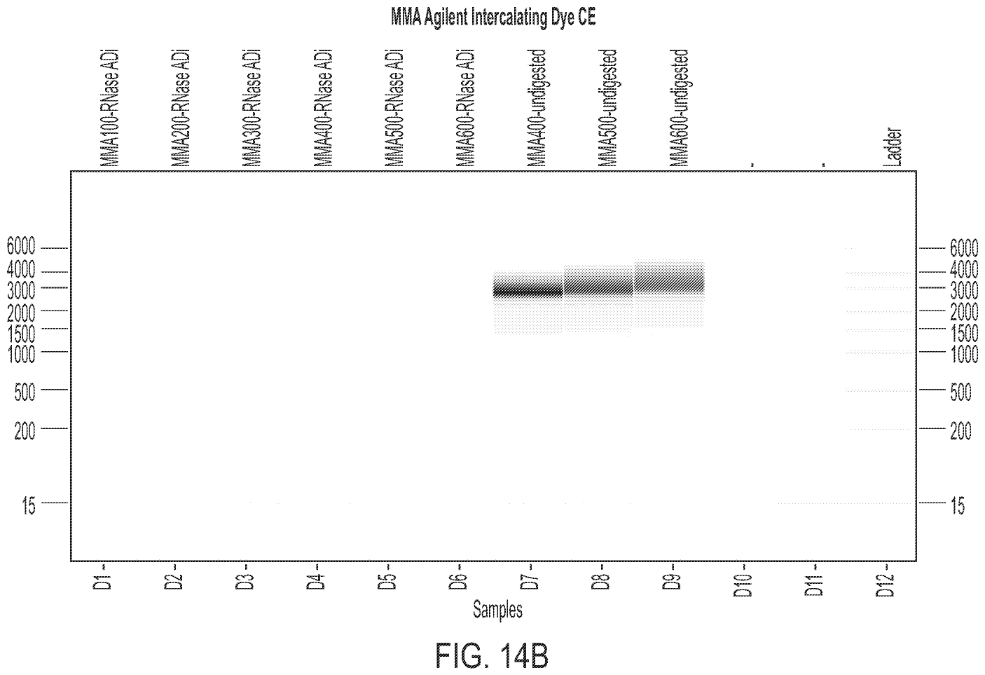

RNase A gel analysis of MMA mRNA comprising poly A tails having poly A tail lengths of 100 nt, 200 nt, 300 nt, 400 nt, 500 nt and 600 nt for Sybr Gold™ dye-treated samples relative to undigested controls having poly A tail lengths of 400 nt, 500 nt and 600 nt is shown in FIG. 14A. RNase A gel analysis of MMA mRNA comprising poly A tails having poly A tail lengths of 100 nt, 200 nt, 300 nt, 400 nt, 500 nt and 600 nt for Agilent™ intercalating dye-treated CE relative to undigested controls having poly A tail lengths of 400 nt, 500 nt and 600 nt is shown in FIG. 14B.

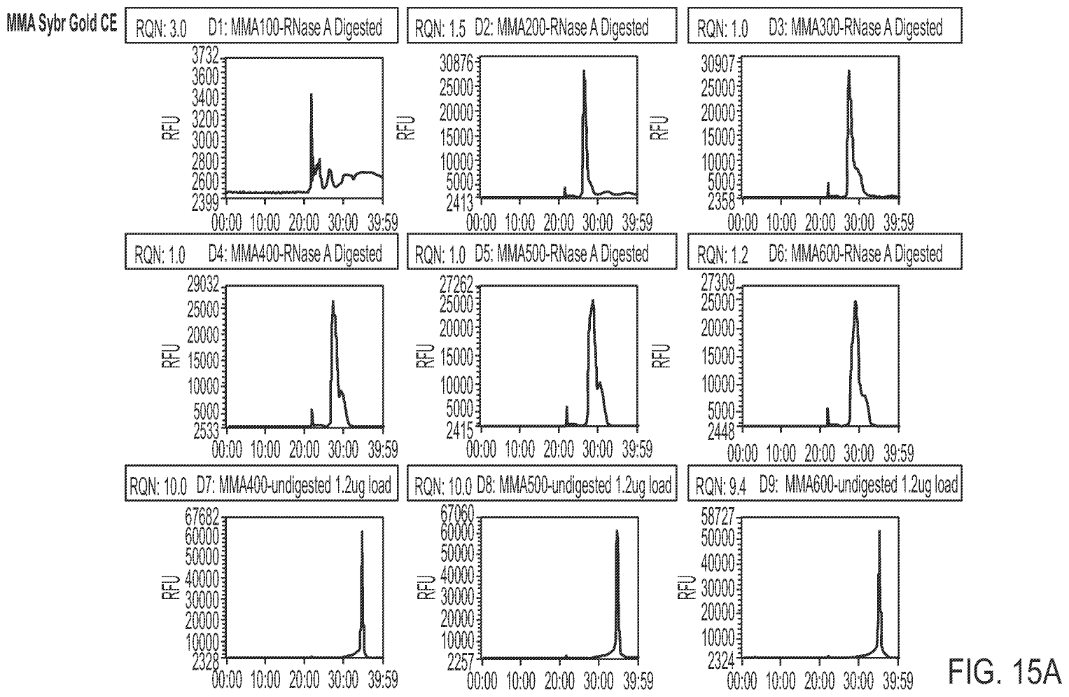

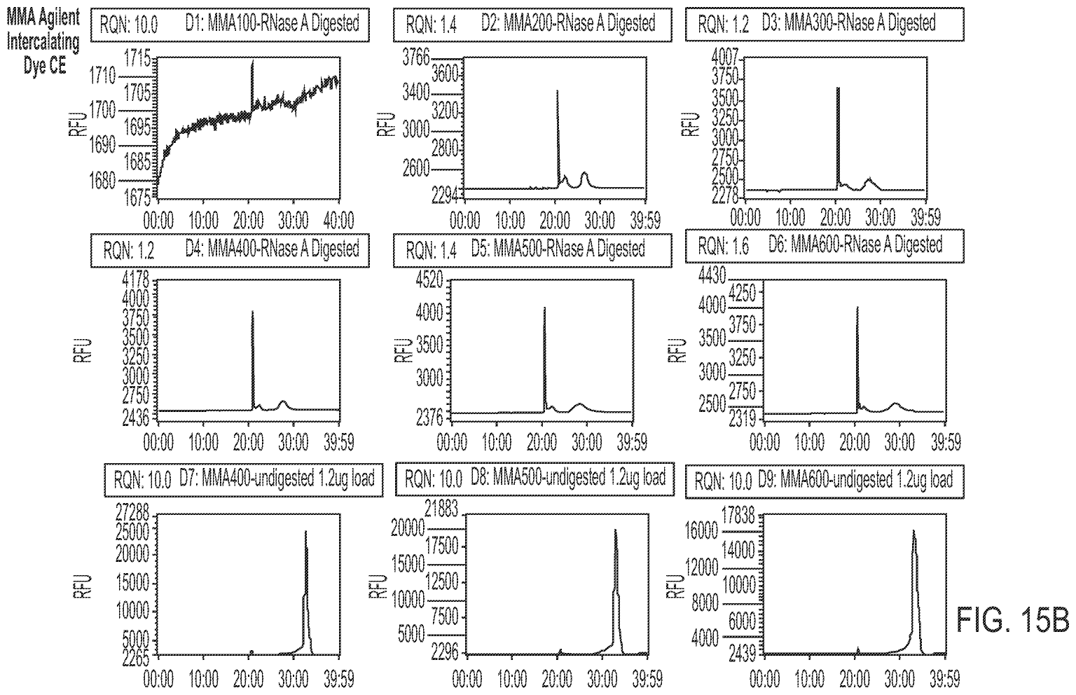

Capillary electropherograms depicting peaks for Sybr Gold™ dye-treated MMA mRNA samples comprising poly A tail lengths of 100 nt, 200 nt, 300 nt, 400 nt, 500 nt and 600 nt relative to undigested controls having poly A tail lengths of 400 nt, 500 nt and 600 nt are shown in FIG. 15A. Capillary electropherograms depicting peaks for Agilent™ intercalating dye-treated MMA mRNA samples comprising poly A tail lengths of 100 nt, 200 nt, 300 nt, 400 nt, 500 nt and 600 nt relative to undigested controls having poly A tail lengths of 400 nt, 500 nt and 600 nt are shown in FIG. 15B. The results show that the signal strength is much lower in the Agilent™ dye-treated samples (FIG. 15B) relative to Sybr Gold™-treated samples (FIG. 15A).

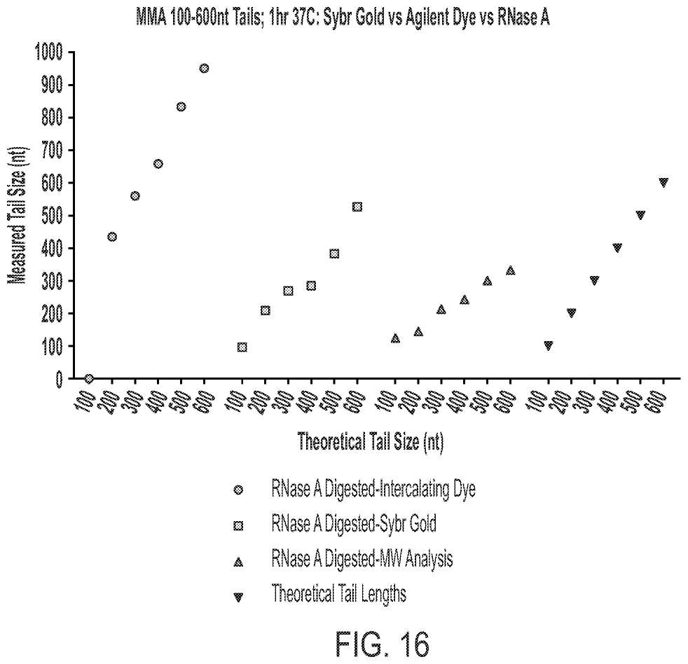

The results from the analysis are shown in a graph of observed tail length on the y-axis and theoretical tail length on the x-axis, for MMA mRNA having poly A tail lengths of 100 nt, 200 nt, 300 nt, 400 nt, 500 nt and 600 nt (FIG. 16). The results are shown for Agilent™ dye-treated samples (plotted from very low raw signals), Sybr Gold™ dye-treated samples, RNase A digested samples followed by Biorad MW analysis and theoretical tail lengths.

As seen in FIG. 16, the observed tail length is underestimated at 100 nt length but overestimated at tail lengths between 200-600 nt in the Agilent™ dye-treated samples, overall resulting in inaccurate poly A tail length measurements.

RNase A digested samples followed by Biorad MW analysis showed accurate tail length measurements comparable to theoretical tail lengths for short tail lengths such as 100 nt. However, between 200-600 nt tail lengths, the observed tail lengths were underestimated relative to theoretical tail lengths.

The Sybr Gold™-treated samples showed accurate tail length measurements between 100 nt-300 nt. Longer tail lengths such as 400 nt-600 nt corresponded more closely to theoretical tail lengths than observed with the RNase A method followed by Biorad MW measurements for tail lengths between 400 nt-600 nt.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of measuring poly A tail length in an mRNA sample, the method comprising:
(a) contacting the mRNA sample with a minor-groove binding dye;
(b) incubating the mRNA sample from (a) with one or more ribonucleases (RNase); and
(c) assaying the sample from (b) by capillary electrophoresis (CE) to determine the poly A tail length of the mRNA.

2. The method of claim 1, wherein the minor-groove binding dye is Sybr Gold™, a Hoechst dye, or 4′,6-diamidino-2-phenylindole (DAPI).

3-4. (canceled)

5. The method of claim 1, wherein the one or more RNases comprise RNaseA and/or RNase T1.

6. The method of claim 1, wherein the CE is coupled with a fluorescence-based detection or UV absorption spectroscopy detection.

7. (canceled)

8. The method of claim 1, wherein incubating the mRNA sample from (a) with one or more ribonucleases (RNase) is for about 15 minutes, 30 minutes, 45 minutes, or 60 minutes.

9. (canceled)

10. The method of claim 1, wherein the poly A tail length is 25 nucleotides or more, 50 nucleotides or more, 100 nucleotides or more, 150 nucleotides or more, or 200 nucleotides or more.

11. The method of claim 1, wherein the poly A tail length is between 50 nucleotides and 5,000 nucleotides, between 100 nucleotides and 1,500 nucleotides, or between 250 nucleotides and 500 nucleotides.

12-14. (canceled)

15. The method of claim 1, wherein one or more steps of the method is automated.

16. The method of claim 15, wherein incubating the mRNA sample from (a) with one or more ribonucleases (RNase) is automated.

17. The method of claim 1, wherein the minor-groove binding dye non-covalently binds to single-stranded RNA (ssRNA).

18. The method of claim 1, wherein the minor-groove binding dye is not an intercalating dye.

19. A method of measuring poly A tail length in an mRNA, the method comprising:
(a) contacting the mRNA sample with Sybr Gold™ minor-groove binding dye;
(b) incubating the mRNA sample from (a) with RNaseA and RNase Tl; and
(c) assaying the sample from (b) by capillary electrophoresis (CE) to determine the poly A tail length of the mRNA.

20. The method of claim 19, wherein the CE is coupled with a fluorescence-based detection or UV absorption spectroscopy detection.

21. (canceled)

22. The method of claim 19, wherein incubating the mRNA sample from (a) with RNaseA and RNase Tl is for about 15 minutes, 30 minutes, 45 minutes, or 60 minutes.

23. (canceled)

24. The method of claim 19, wherein the poly A tail length is 25 or more nucleotides, 50 or more nucleotides, 100 or more nucleotides, 150 or more nucleotides, or 200 or more nucleotides.

25. The method of claim 19, wherein one or more steps of the method is automated, and/or wherein the method is high throughput.

26. (canceled)

27. A method of measuring homopolymeric nucleotide length in an mRNA sample, the method comprising:
(a) contacting the mRNA sample with a minor-groove binding dye;
(b) incubating the mRNA sample from (a) with one or more ribonucleases (RNase); and
(c) assaying the sample from (b) by capillary electrophoresis (CE) to determine the homopolymeric nucleotide length of the mRNA.

28. The method of claim 27, wherein the homopolymeric nucleotide length is 25 nucleotides or more, 50 nucleotides or more, 100 nucleotides or more, 150 nucleotides or more, or 200 nucleotides or more.

29. The method of claim 27, wherein the homopolymeric nucleotide length is between 50 nucleotides and 5,000 nucleotides.

30. (canceled)

31. The method of claim 27, wherein the nucleotides comprising the homopolymeric nucleotide is selected from A, U, G, or C.


SEQUENCE LISTING (3 sequences)

SEQ ID NO: 1 - DNA, 23 bp, T3 bacteriophage
aattaaccctcactaaagggaga

SEQ ID NO: 2 - DNA, 23 bp, T7 bacteriophage
taatacgactcactatagggaga

SEQ ID NO: 3 - DNA, 23 bp, SP6 bacteriophage
atttacgacacactatagaagaa